(19) United States
(12) Patent Application Publication
(10) Pub. No.: US 2010/0016906 A1
(43) Pub. Date: Jan. 21, 2010

(54) DEVICE AND METHOD TO ACCESS THE ANTERIOR COLUMN OF THE SPINE

(76) Inventor: M. Samy Abdou, San Diego, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/507,003

(22) Filed: Jul. 21, 2009

Related U.S. Application Data
(60) Provisional application No. 61/135,472, filed on Jul. 21, 2008.
(60) Provisional application No. 61/207,535, filed on Feb. 14, 2009.

(51) Int. Cl. A61B 17/58

(52) U.S. Cl. 606/86 R

(57) ABSTRACT

Devices and methods for delivery of inter-vertebral implants that do not require extensive dissection of normal tissues or significant retraction of the nerve elements are disclosed. The devices and methods provide ease of use as well as a safe and familiar surgical approach that maximizes the likelihood of optimal device placement within the inter-vertebral space.

### REFERENCE TO PRIORITY DOCUMENTS

This application claims priority of co-pending U.S. Provisional Patent Application Ser. No. 61/135,472 filed Jul. 21, 2008 and U.S. Provisional Patent Application Ser. No. 61/207,535 filed Feb. 14, 2009. Priority of the aforementioned filing dates is hereby claimed and the disclosure of each Provisional patent applications is hereby incorporated by reference in its entirety.

### BACKGROUND

Surgical reconstructions of the bony skeleton are common procedures in current medical practice. Regardless of the anatomical region or the specifics of the reconstructive procedure, many surgeons employ an implantable device between bony segments in order to adjust, align and maintain the spatial relationship(s) between them.

Placement of an inter-vertebral device within the spine may be performed through various approaches. Access to the anterior aspect of the spine provides a direct route for device placement. However, since the spine is situated posteriorly within the body cavity, an anterior approach requires dissection through the many vital tissues that lie anterior to the spine. Likewise, a lateral approach also requires extensive dissection of the body cavity. Both approaches are more difficult in the thoracic and lumbar spine, since these body cavities contain far more tissue anterior and lateral to the spine.

A posterior approach provides ready access to the posterior aspect of the spine through an operative corridor that is familiar to all spine surgeons. Unfortunately, the nerve elements are situated posterior to the inter-vertebral space and will limit access to that space. Hence, use of the posterior approach for the placement of any sizable device within the inter-vertebral space risks permanent neurological injury.

### SUMMARY

In view of the proceeding, there is a need for devices and methods for delivery of inter-vertebral implants that do not require extensive dissection of normal tissues or significant retraction of the nerve elements. The devices and methods desirably provide ease of use as well as a safe and familiar surgical approach that maximizes the likelihood of optimal device placement within the inter-vertebral space.

In one aspect, there is disclosed a method for performing a procedure on a segment of a vertebral column of a subject, wherein the segments include at least two vertebral bones and an intervening disc space, comprising: identifying a spinal disc space within the spinal segment using radiographic imaging, wherein the identified disc space is located between adjacent vertebrae and has an anterior aspect, a posterior aspect, a first side aspect and a second side aspect; penetrating the skin of the subject at a position that is posterior to and lateral to the tip of the transverse process of the target spinal segment; advancing an insertion member through the site of skin incision, wherein the insertion member comprises a curved elongate body that has a proximal end and a distal end, wherein the curved elongate body contains a curved outer tract that extends from a proximal end of the curved elongate body to a distal end of the curved elongate body; forming an arcuate pathway from the skin incision site through tissues exterior to the vertebral column until the opening in the distal surface of the curved elongate body abuts the first side aspect of the target spinal segment; coupling one end of a mount to the proximal end of the curved elongated body and another end of the mount to a surface with a defined spatial relationship to the identified disc space, wherein the coupled mount limits the movement of the insertion device relative to the disc space; and advancing an orthopedic device along the curved outer tract of the curved body member and onto the target spinal segment of the vertebral column.

Other features and advantages should be apparent from the following description of various embodiments, which illustrate, by way of example, the principles of the disclosed devices and methods.

### BRIEF DESCRIPTION OF THE DRAWINGS

FIG. 1 illustrates multiple views of a vertebral bone.

FIGS. 2 A and 2B show views of two vertebral bones in a functional spinal unit.

FIG. 3 illustrates a cross sectional view of the torso at the level of the lumbar spine.

FIG. 4 shows a schematic representation of the posterior aspect of a patient that is positioned prone.

FIG. 5 shows an instrument placed into the disc space.

FIG. 6 illustrates perspective views of the instrument.

FIG. 7 illustrates orthogonal views of the instrument.

FIGS. 8A-9B show additional views of the instrument.

FIG. 10 shows the instrument in the disc space

FIGS. 11A-11B illustrates perspective views of an alternate embodiment of an instrument.

FIG. 12 illustrate a radial cross sectional view of a device within a tract.

FIG. 13 shows an example of a bone screw assembly.

FIGS. 14-21 show various views of a platform and exemplary method of use.

FIG. 22 shows a completed construct.

FIGS. 23A-26 show an implant placement instrument and associated method of use.

FIG. 26 illustrates an additional method of implant placement.

FIGS. 27 to 32B illustrate an additional embodiment.

FIG. 33-36A illustrates an embodiment of a disc space prosthesis adapted to preserve motion between the two vertebras and associated method of use.

FIG. 37A illustrates a device adapted to provide an immediate replacement bearing surface within a damaged disc.

FIG. 37B illustrates a cylindrical implant with a central bore.

FIG. 38A shows an X-ray from an actual patient, wherein the lumbar vertebrae have assumed an abnormal, scoliotic alignment as a direct result of the artificial disc implantation.

FIG. 38B shows a schematic representation of the scoliotic deformity.

FIG. 39-43 show various views of a device adapted to replace the function of an inter-vertebral disc.

FIG. 44 illustrates the device of FIG. 39 after lateral insertion into a disc space.

FIGS. 45A to 49B show a disc prosthesis that may be repositioned within the disc space after insertion.

### DETAILED DESCRIPTION

In order to promote an understanding of the principals of the disclosure, reference is made to the drawings and the embodiments illustrated therein. Nevertheless, it will be understood that the drawings are illustrative and no limitation of the scope of the invention is thereby intended. Any such alterations and further modifications in the illustrated embodiments, and any such further applications of the principles of the invention as illustrated herein are contemplated as would normally occur to one of ordinary skill in the art.

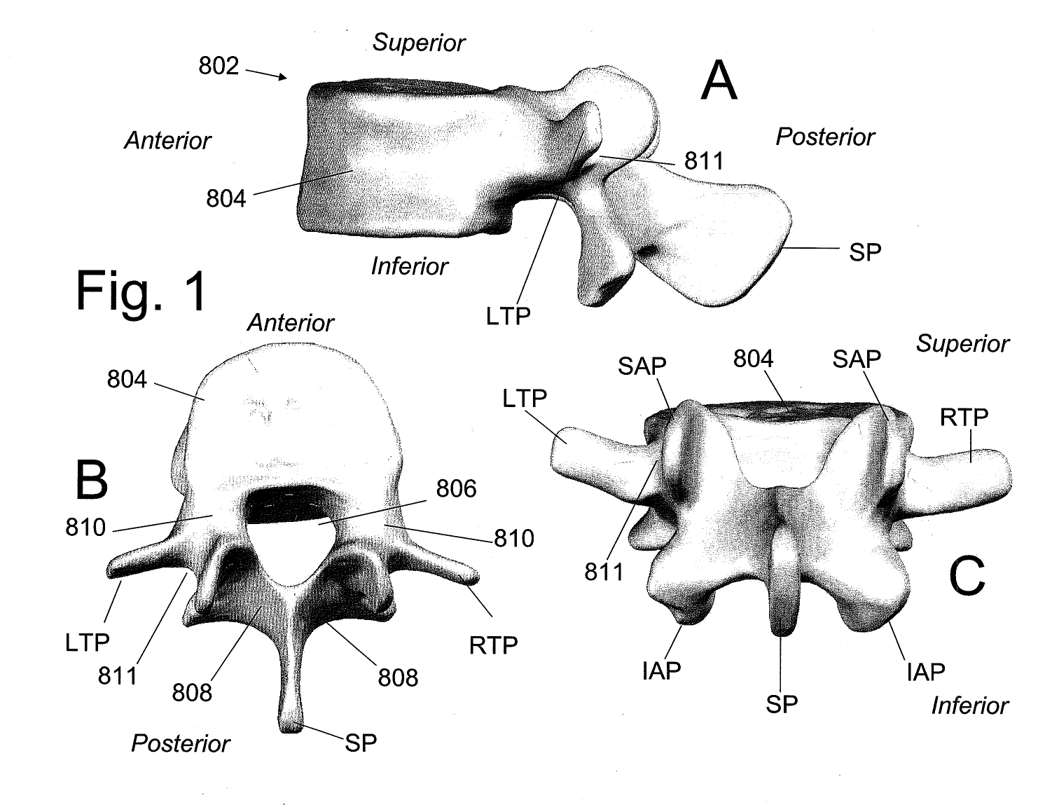

FIG. 1 shows a diagrammatic representation of a spinal vertebral bone 802 in multiple views. For clarity of illustration, the vertebral bone of FIG. 1 and those of other illustrations presented in this application are represented schematically and those skilled in the art will appreciate that actual vertebral bodies may include anatomical details that are not shown in these figures. Further, it is understood that the vertebral bones at a given level of the spinal column of a human or animal subject will contain anatomical features that may not be present at other levels of the same spinal column. The illustrated vertebral bones are intended to generically represent vertebral bones at any spinal level without limitation. Thus, the disclosed devices and methods may be applied at any applicable spinal level.

Vertebral bone 802 contains an anteriorly-placed vertebral body 804, a centrally placed spinal canal and 806 and posteriorly-placed lamina 808. The pedicle (810) segments of vertebral bone 802 form the lateral aspect of the spinal canal and connect the laminas 808 to the vertebral body 804. The spinal canal contains neural structures such as the spinal cord and/or nerves. A midline protrusion termed the spinous process (SP) extends posteriorly from the medial aspect of laminas 808. A protrusion extends laterally from each side of the posterior aspect of the vertebral bone and is termed the transverse process (TP). A right transverse process (RTP) extends to the right and a left transverse process (LTP) extends to the left. A superior protrusion extends superiorly above the lamina on each side of the vertebral midline and is termed the superior articulating process (SAP). An inferior protrusion extends inferiorly below the lamina on each side of the vertebral midline and is termed the inferior articulating process (IAP). Note that the posterior aspect of the pedicle can be accessed at an indentation 811 in the vertebral bone between the lateral aspect of the SAP and the medial aspect of the transverse process (TP). In surgery, it is common practice to anchor a bone fastener into the pedicle portion of a vertebral bone by inserting the fastener through indentation 811 and into the underlying pedicle.

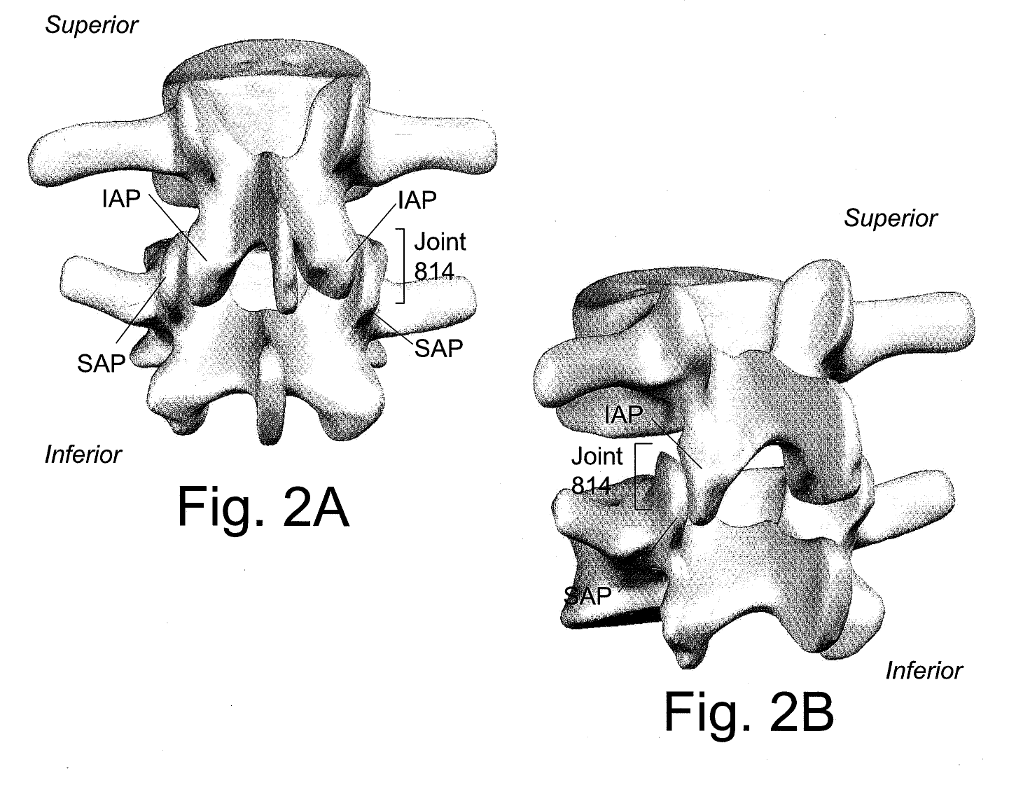

FIGS. 2A and 2B illustrate a functional spinal unit (FSU), which includes two adjacent vertebrae and the intervertebral disc between them. The intervertebral disc resides between the inferior surface of the upper vertebral body and the superior surface of the lower vertebral body. (Note that a space is shown in FIGS. 2A and 2B where intervertebral disc would reside.) FIG. 2A shows the posterior surface of the adjacent vertebrae and the articulations between them while FIG. 2B shows an oblique view. Note that FSU contains a three joint complex between the two vertebral bones, with the intervertebral disc comprising the anterior joint. The posterior joints include a facet joint 814 on each side of the midline, wherein the facet joint contains the articulation between the IAP of the superior vertebral bone and the SAP of the inferior bone.

The preceding illustrations and definitions of anatomical structures are known to those of ordinary skill in the art. They are described in more detail in Atlas of Human Anatomy, by Frank Netter, third edition, Icon Learning Systems, Teterboro, N.J. The text is hereby incorporated by reference in its entirety.

Disclosed are methods and devices that permit a surgeon to access the anterior column of the spine from a posterior approach without significant manipulation of the intervening nerve elements. (The “anterior column” is used here to designate that portion of the vertebral body and/or FSU that is situated anterior to the posterior longitudinal ligament. Thus, its use in this application encompasses both the anterior and middle column of Denis. See The three column spine and its significance in the classification of acute thoracolumbar spinal injuries. By Denis, F. Spine 1983 November-December; 8(8):817-31. The article is incorporated by reference in its entirety.)

A skin incision is placed lateral and posterior to the tip of the transverse process of a target spinal vertebra of a functional spinal unit and a curvilinear port is advanced into the subject's body through that skin incision. The port is guided through the para-spinal soft tissues and onto the lateral aspect of the adjacent disc space and/or vertebral body of the target spinal vertebra —wherein, the distal aspect of the curvilinear port is situated anterior and medial to the tip of the transverse process. In an embodiment, there is provided a method for placement of the port using free-hand guidance and tactile feel by the surgeon based on radiographic visualization. The port is not positioned by a guide or platform that has already been inserted into the body prior to port placement. In an embodiment, after post placement, the aspect of the port that rests outside the body is attached to the platform so as restrain the movement of the port relative to the target spinal vertebra. In an embodiment, the attachment platform is affixed to a second portion of the target spinal vertebra.

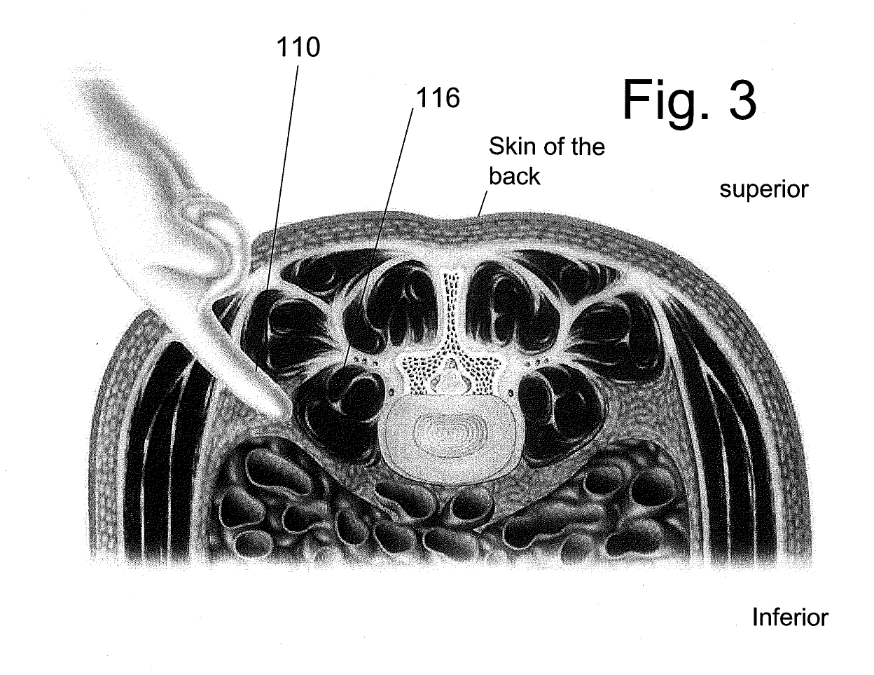

FIG. 3 illustrates a cross sectional view of the torso at the level of the lumbar spine. For clarity of illustration, the contents are represented schematically and those skilled in the art will appreciate that an actual cross section of the human torso may include anatomical details not shown in FIG. 3.

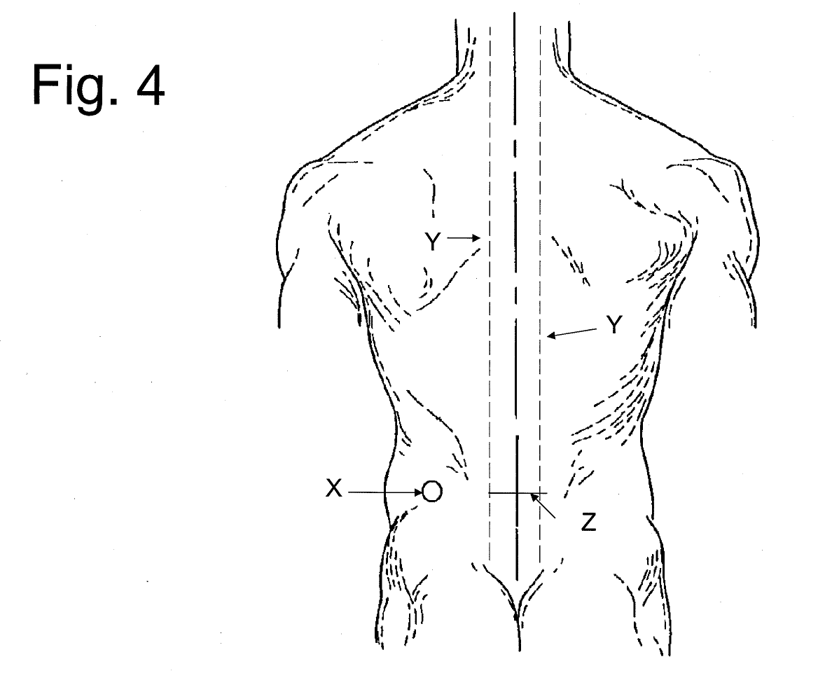

In preparation for percutaneous placement of an orthopedic implant into a spinal disc space, the patient is preferably placed in a prone position with spine 102 and skin 105 in the superior position (as shown in FIG. 3). The level of the spine that is to be implanted is localized on X-ray in at least one plane. After the customary sterile preparation of the operative site, the surgeon localizes an incision point on the skin that is lateral and posterior to the transverse process of the spinal segment that will be accessed. Preferably, the incision is lateral to the paraspinal muscles (the erector spinae muscle group and/or others). However, the incision is not placed directly lateral to the side of the disc space. FIG. 4 shows a schematic representation of the posterior aspect of a patient that is positioned prone. The skin overlying the back is shown. Lines Y show the lateral extent of the transverse processes of the spinal column. Assuming that the spinal level to be accessed is at line Z, the surgeon makes an incision at or about point X.

At least one finger 110 is placed into the retro-peritoneal space and the lateral aspect of the psoas muscle 116 is palpated, as shown in FIG. 3. In this way, the surgeon can directly access the psoas muscle (and the lateral aspect of the disc space) using tactile feel. Instrument 130 is then guided by the surgeon along the finger 110 and onto the lateral aspect of the psoas muscle 116. The position of instrument 130 may be verified by x-ray examination. (While less secure that the method described above, the surgeon can alternatively advance instrument 130 directly through the skin incision, through the para-spinal tissue and onto the lateral aspect of the psoas muscle without prior digital palpation of the muscle.) No placement instrument or platform is used to perform the initial positioning of instrument 130 within the subject's body.

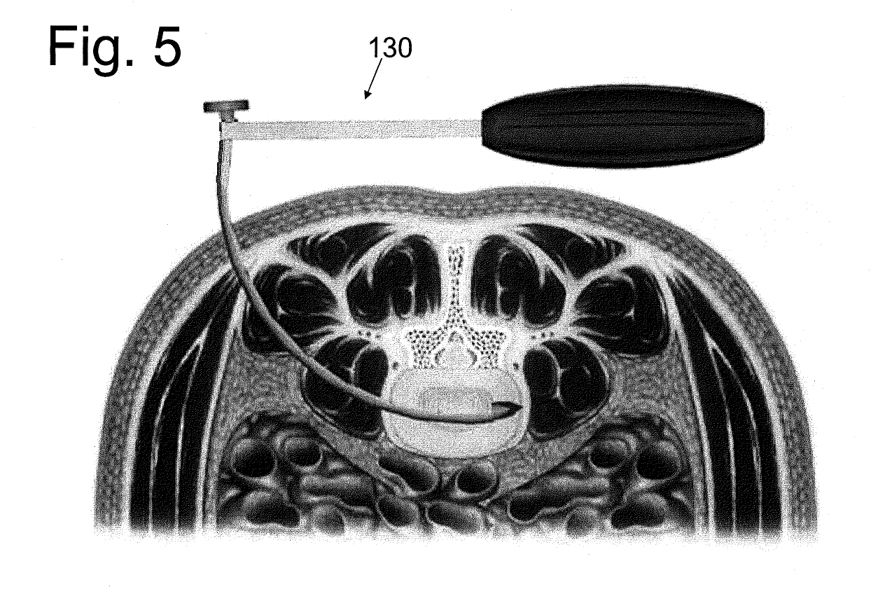

Instrument 130 is advanced through the psoas muscle into the disc space. Since important nerve structures may transverse the psoas, instrument 130 (and/or a probe or device placed through the channel of instrument 130) is preferably connected to an Electromyography (EMG) apparatus (or any other electrical system that is used to localize nerve tissue), and used, at least partially, as an EMG probe during advancement through the muscle. In this way, the advancement of instrument 130 through the psoas muscle is performed under EMG guidance. Under X-ray visualization, instrument 130 is placed into the disc space as shown in FIG. 5. Instrument 130 has a convex lateral surface that functions as a tract along which additional instruments may be passed to manipulate the target spinal segment and/or implant device and substances at the targeted site.

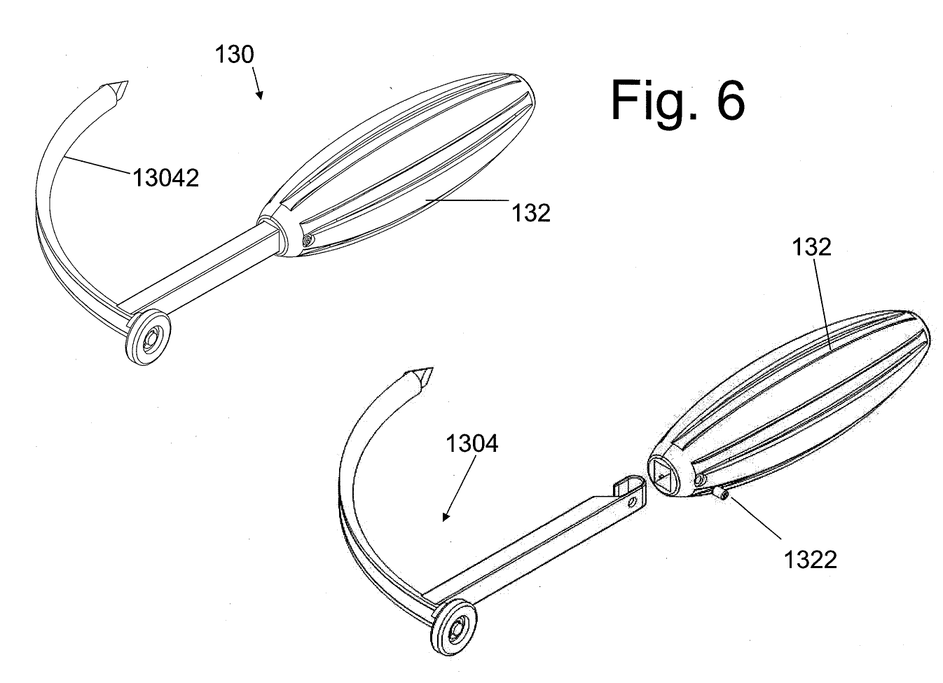

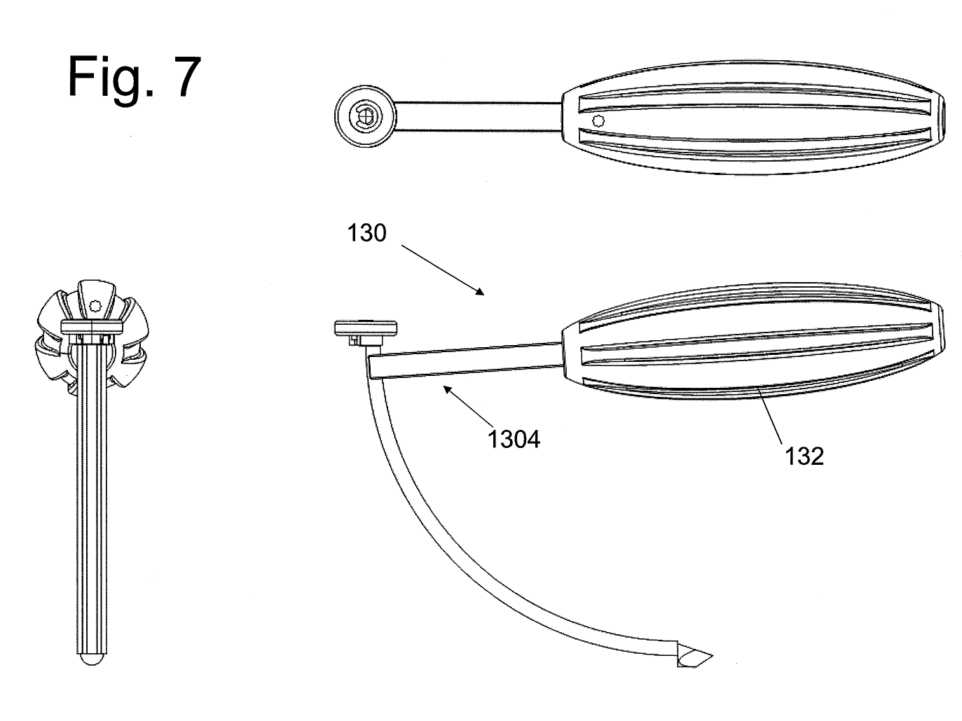

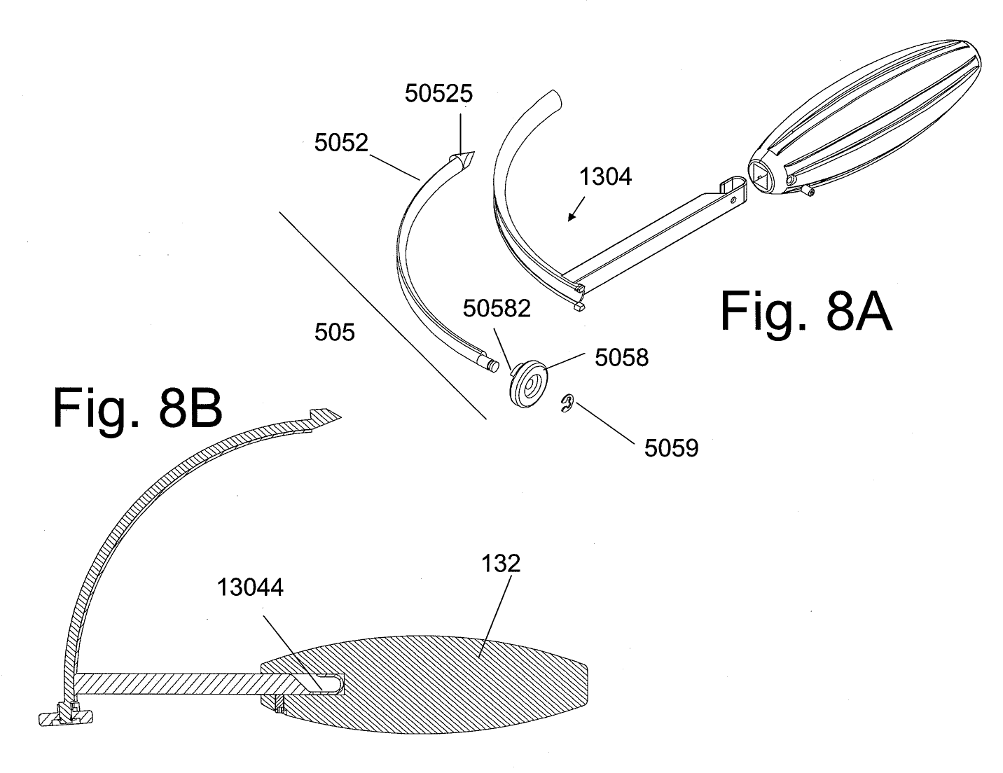

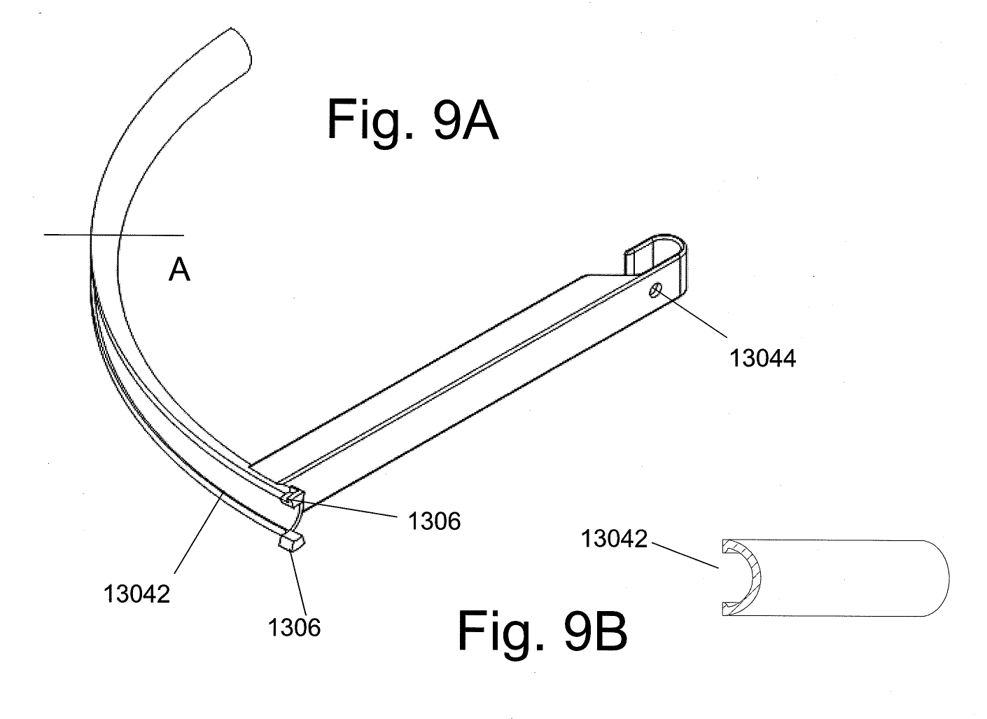

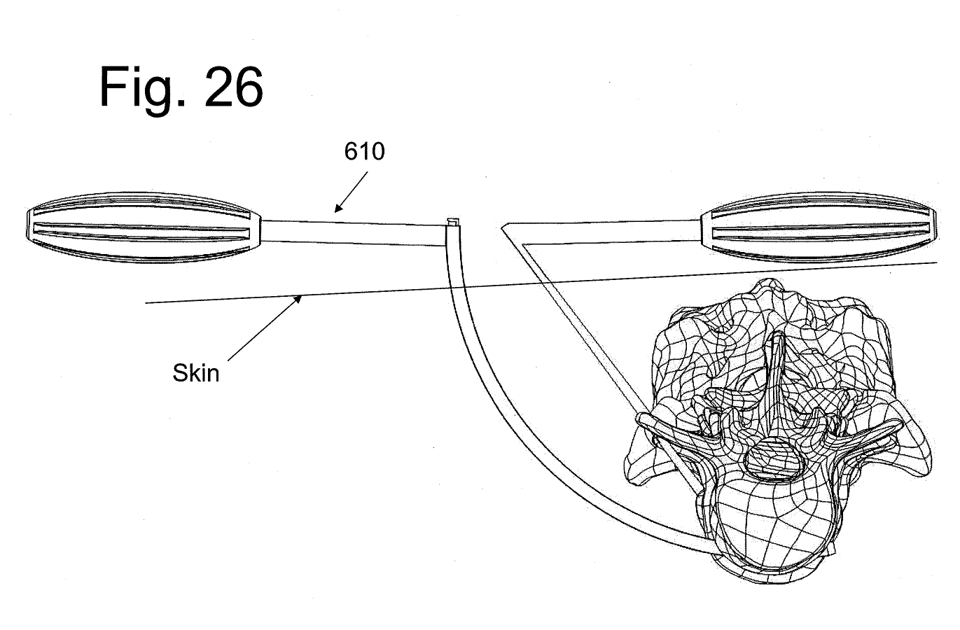

FIG. 6 illustrates perspective views of instrument 130 while orthogonal views are shown in FIG. 7. A disassembled view is shown in FIG. 8A. Instrument 130 is comprised of a handle member 132 and member 1304 with curvilinear tract 13042. Handle 132 is removably attached to member 1304 and may be locked to member 1304 by threaded set screw 1322. While the curvature of tract 13042 is depicted as convex, the tract may be concave (see FIG. 26). As shown in FIG. 9A, protrusions 1306 are positioned at the proximal aspect of tract 13042 and adapted to provide an anchor site onto instrument 130 for devices guided to the target site within a subject by tract 13042. Member 5052 of device 505 is adapted to at least partially reside within tract 13042 and has a pointed tip 50525 that is adapted to divide tissue and permit the advancement of assembled instrument 130. Member 5058 of device 505 contains protrusions 50582 that are adapted to interact with complimentary protrusions 1306 of device 130 and anchor instrument 505 to device 130. Clip 5059 of device 505 keeps member 5058 attached to member 5052. A sectional view is shown in FIG. 8B.

FIG. 9A shows the member 1304. FIG. 9B illustrates a radial cross sectional view of tract 13042, wherein the section is taken along plane A of FIG. 9A. Threaded bore hole 13044 is adapted to accept a threaded set screw.

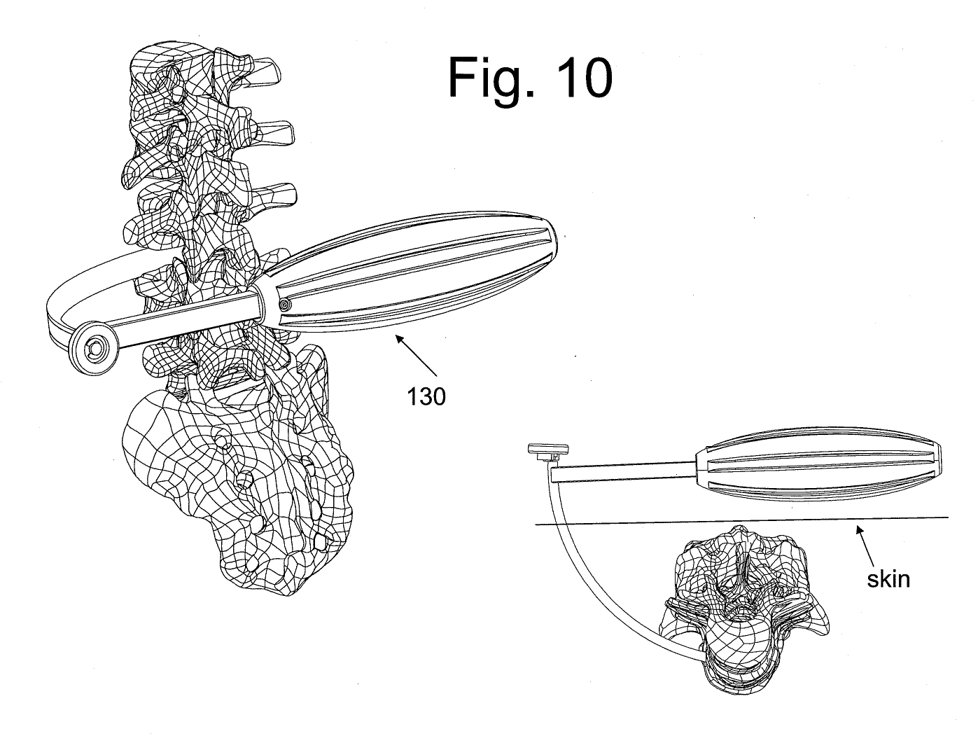

Instrument 130 may be held using handle 132 and the totality of the implantation procedure may be performed while instrument 130 is hand-held. Alternatively, instrument 130 may be coupled to a platform that is anchored into the spinal bone, thereby forming a substantially rigid jig for implant placement. FIG. 10 shows instrument 130 in the disc space. Device 505 is removed and the curvilinear channel 13042 of instrument 130 is used to perform work on the disc space and/or vertebral bone. Instrument 130 may be also used to position any substance or device adapted for biological implantation into the spinal disc/vertebra or onto the exterior aspect of them. By way of example, the implant may include a prosthesis adapted to fuse spinal segments, replace the bone segments, and/or replace the natural function of the inter-vertebral disc.

In addition, substances adapted to regenerate, replace and/or rejuvenate the function of the damaged disc may be instilled directly into the intervertebral disc, vertebral bone, annulus fibrosis and/or adjacent ligament structures. Such substances include, but are not limited to, one or more of the following: living or non-living biological cells, genetically engineered/altered genetic vectors (such as viruses and the like), extracts of biological tissues, natural or synthetic materials adapted to function as malleable shock absorber, natural or synthetic frameworks adapted to promote cell adhesion and/or growth, connective tissue matrices, nutrient-containing media, growth factors and the like.

It should be appreciated that instrument 130 may be alternatively used to perform surgical work upon the vertebral bones directly, such as perform partial or total corpectomy, and to apply a prosthesis and/or biological materials onto the lateral aspect of the spine.

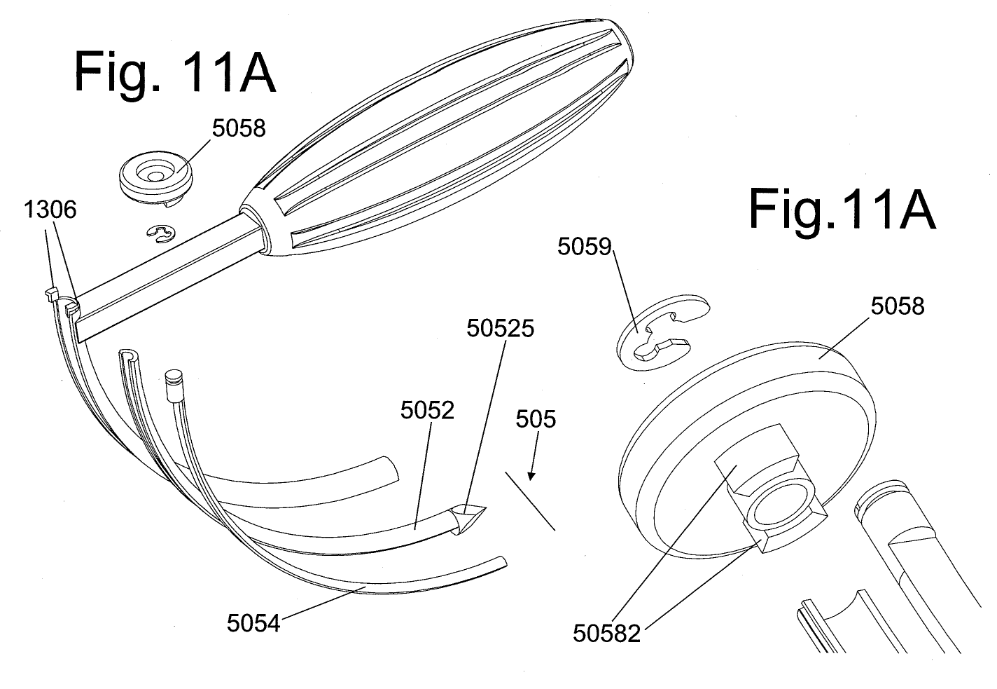

In an additional embodiment, instrument 130 may be adapted to contain a balloon within device 505. The balloon may be used to create cavities and/or channels within the tissues within the body of the subject. FIGS. 11A-11B illustrates perspective views of instrument 130 (with balloon). Instrument 130 is comprised of a handle member 132 and curvilinear tract 1304. While the curvature of tract 1304 is depicted as convex and coplanar relative to handle 132, the tract may be concave (see FIG. 26). As shown in FIG. 11A, protrusions 1306 are positioned at the proximal aspect of tract and adapted to provide an anchor site onto instrument 130 for devices guided to the target site within a subject by tract 1304. Device 505 is comprised of a first member 5052 that is adapted to at least partially reside within tract 1304, a second member 5054 that is adapted to at least partially reside within member 5052, a balloon member 5056 that is retained by the interaction of members 5052 and 5054. A member 5058 is attached to the proximal end of member 5054 and retained in the assembled position by ring 5059 (exploded view in FIG. 11A). Member 5058 contains protrusions clip 50582 that are adapted to interact with complimentary protrusions 1306 of device 130 and anchor instrument 505 to device 130.

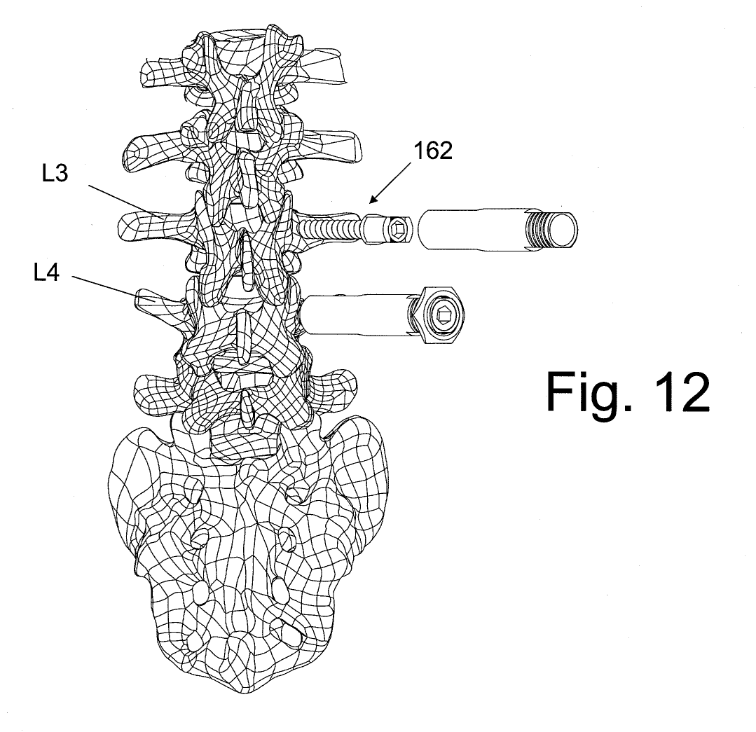

FIG. 12 illustrate a radial cross sectional view of device 505 within tract 1304. In FIG. 12A, balloon member 5056 is deflated, wherein FIG. 12B shows balloon member 5056 inflated. Member 5052 has outer indentation that interacts with a complimentary protrusion of tract 1304 and retains device 505 within the tract. In cross section, members 5052 and 5054 form portions of concentric circles so that member 5054 is contained within member 5054 and can not dissemble. The distal end 50525 of member 5052 contains a cutting point that is adapted to cut tissue. Balloon member 5056 has flap 50562 that is adapted to at least partially surround member 5054 and be solidly retained between members 5052 and 5054 within device 505. In assembly of device 505, flap 50562 (which may be alternatively made a two or more sub-segments) is slipped over member 5054 and the latter is placed within the internal cavity of member 5052. The distal aspect of member 5054 is retained within a pocket within distal end 50525 and the proximal aspect of member 5054 is attached to the proximal aspect of member 5052 by the action of member 5058. In this way, device 505 is retained in the assembled state.

In use, device 505 is inserted through instrument 130 and into the disc space. The balloon may be deployed to expand anteriorly (away from the spinal canal) or posteriorly (towards the spinal canal). Depending on the balloon location along device 505, it can be used to divide the tissues and develop a cavity along any segment extending from the skin surface to the disc space. The inflated balloon diameter can vary and be used to generate cavities of varying diameters.

Preferably, but not necessarily, the balloon member 5056 is positioned across the total disc space prior to inflation so that the corridor transverses the disc space. The balloon may be repeatedly inflated/deflated until the tissue corridor is sufficiently established. Device 505 is removed after the final balloon deflation and instrument 130 is then used to deliver the tools for the remaining portion of the procedure as previously described.

As noted, instrument 130 may be alternatively coupled to a platform that is anchored into the spinal bone, thereby forming a substantially rigid jig onto which instrument 130 may be attached. FIG. 12 shows bone fasteners being affixed to the pedicle potion of bone. Those skilled in the art will appreciate that the actual vertebral bones may include anatomical details not shown in FIG. 12. A bone screw assembly 162 that contains at least one connecting rod-receptacle member 164 and bone screw 166 is shown anchored to bone. In the illustration, the upper screw is affixed into the L3 lumbar vertebra and the lower screw is affixed to the L4 lumbar vertebra.

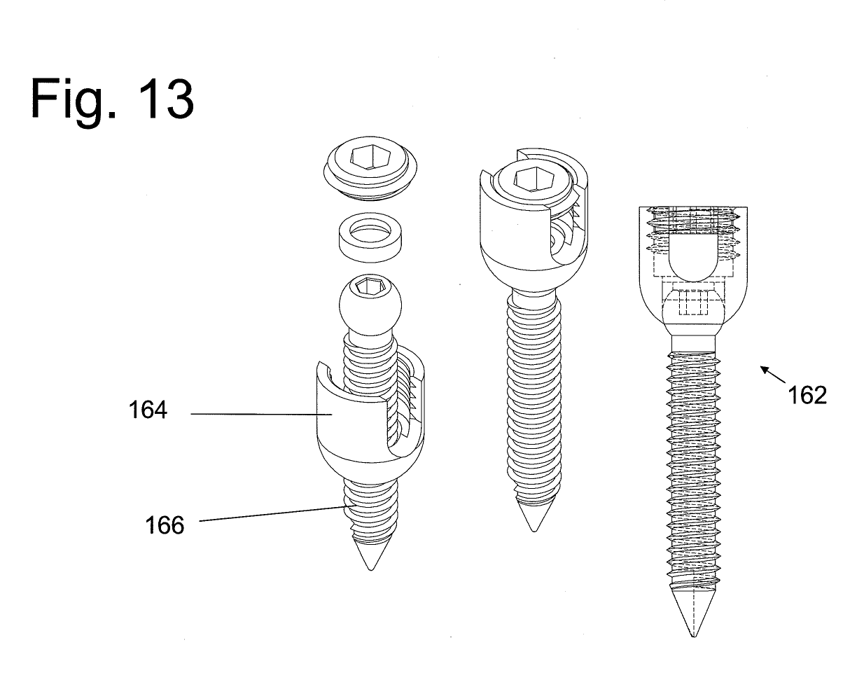

An example of a bone screw assembly 162 is shown in FIG. 13. Preferably, assembly 162 can reversibly transition from a first state, wherein the rod-receptacle member is freely movable relative to the screw shank, to a second state wherein the rod-receptacle member is rigidly affixed to the screw shank. By way of an example, U.S. Pat. No. 5,672,176 discloses a version of a poly-axial bone screw. The text is hereby incorporated by reference in its entirety. Alternative bone fasteners are well known in the art and any of these screw embodiments may be alternatively used.

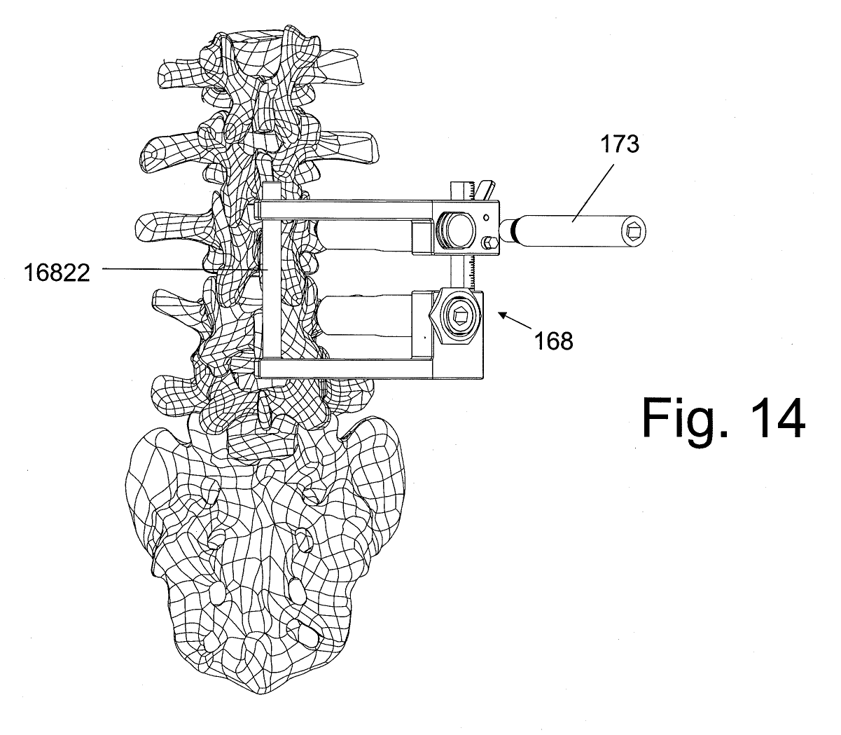

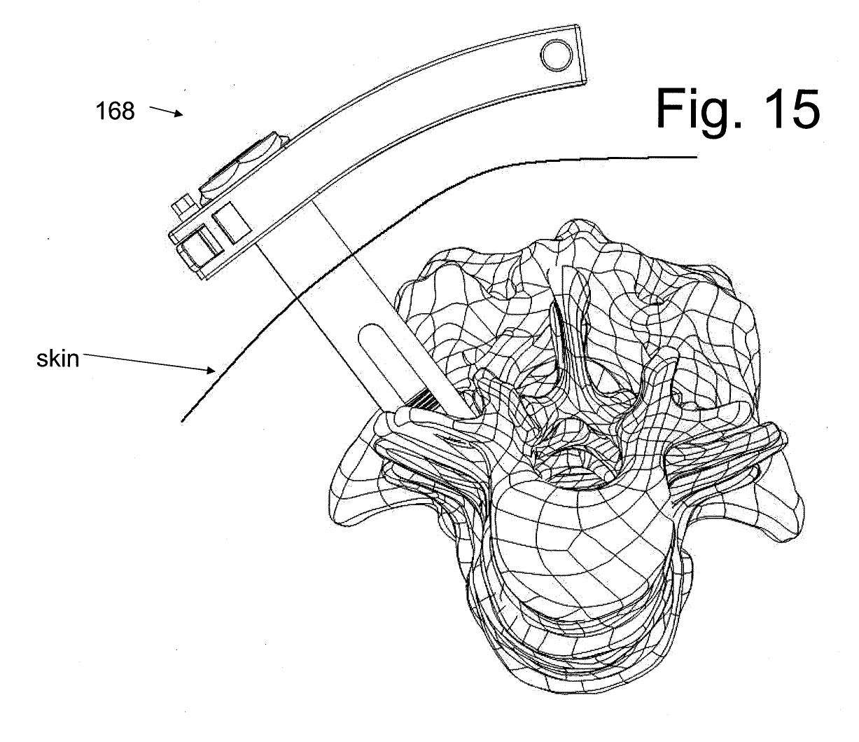

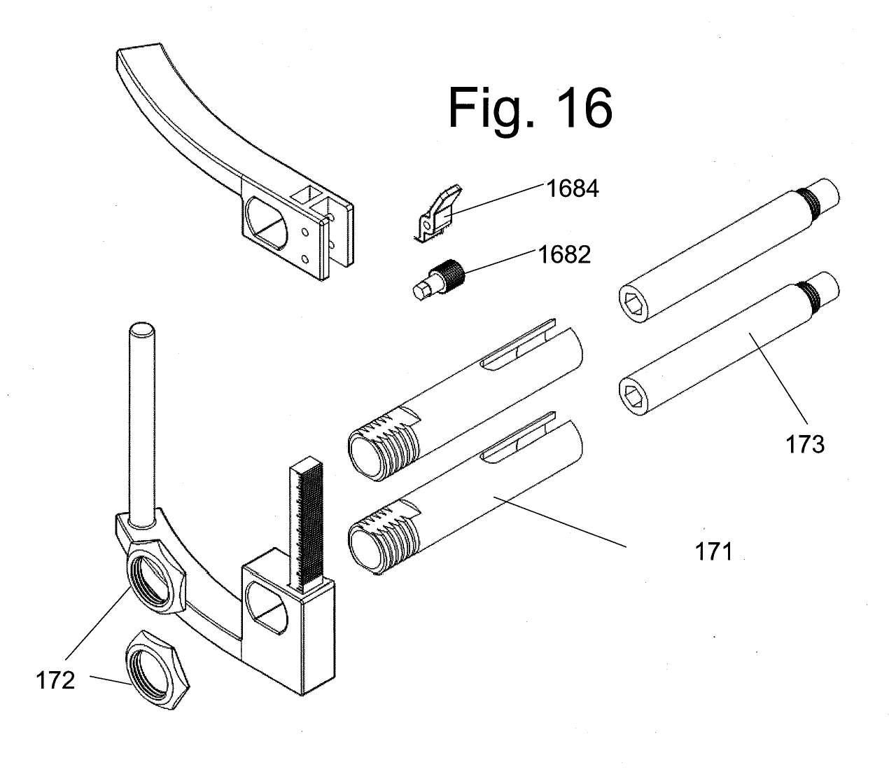

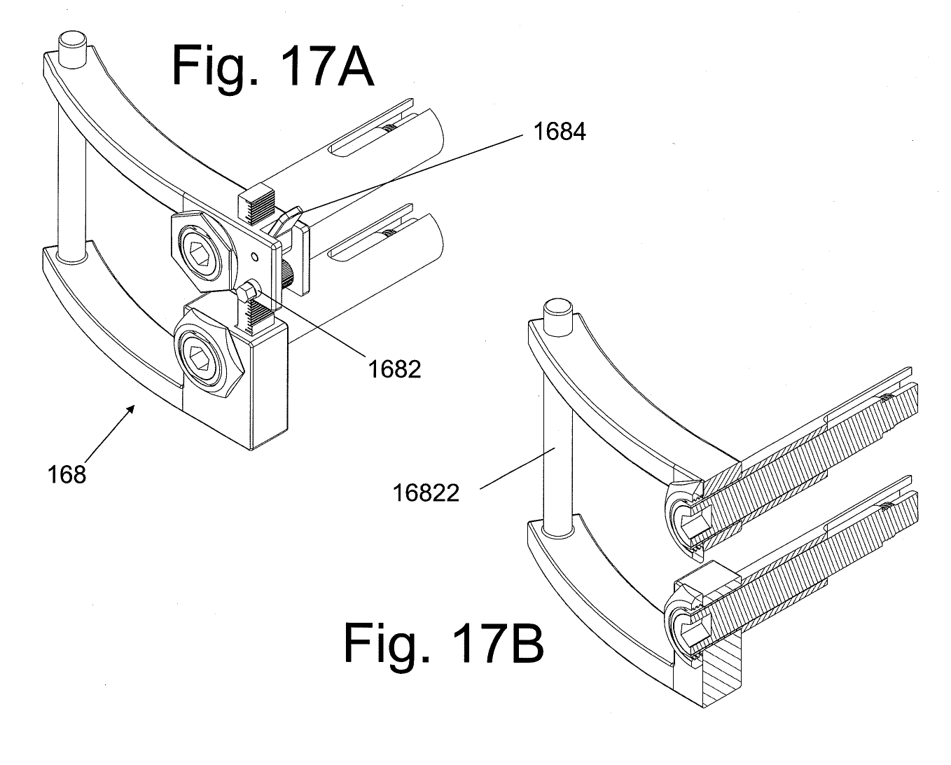

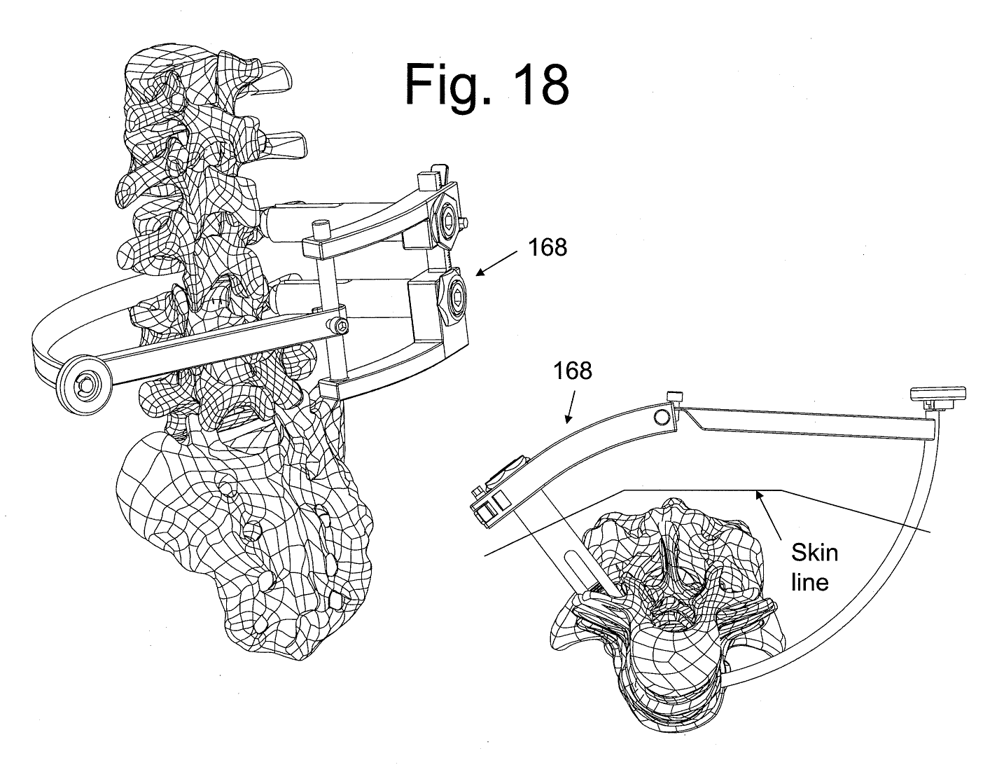

A rod-receptacle member 164 is rigidly attached to each screw assembly 162 and is then coupled to platform 168. The platform is illustrated in an exploded view in FIG. 16. It is shown in an assembled view in FIG. 17A and a cross sectional view is shown in FIG. 17B. FIGS. 14 and 15 illustrate the platform in various stages of attachment onto the bone screw assemblies 162. Note that the platform 168, once fully affixed to assemblies 162 (seen in FIG. 18), provides a rigidly fixed platform for attachment of instrument 130 relative to the vertebral bone. The platform also permits distraction of the bone screw assemblies 162 by the actuation of member 1682. Pawl 1684 retains the distracted platform in the distracted position.

While briefly described in the preceding section, it is understood that the disclosed distraction platform is illustrative. Multiple alternative embodiments of distraction platforms are known in the art and it is contemplated that other platforms may be alternatively employed. For example, US Patent Application Publication number 2005/0021040 discloses one of many such distraction platform. (The application is incorporated by reference in its entirety.) The platform may be modified for use in the current application.

In the method of use wherein instrument 130 is coupled to a platform, it is preferable, but not necessarily, to affix the bone screw assemblies and platform to the vertebral bones prior to placement of instrument 130 into the body of the subject. In reference to FIG. 14, bone screw assemblies 162 are advanced through the skin and into the pedicle portion of bone at or about point 811 (See FIG. 1) of the vertebra L3 and vertebra L4. This is preferably performed under X-ray guidance and using percutaneous methods that are well known to those in the art. Guide tubes 171 are used to place assemblies 162 through the skin and to interconnect them to the platform 168 after the assemblies are attached to bone. The platform is subsequently seated onto the proximal ends of tubes 171 (shown with threads in FIG. 16) and nuts 172 are used to rigidly affix the platform to the tubes.

At this point, each tube 171 is rigidly attached to platform 168 at a proximal end and to the rod-receptacle member 164 of screw assembly 162 at a distal end. Each rod-receptacle member 164 remains mobile relative to the bones screws 166 of its screw assembly 162. The platform 168 is moved under x-ray guidance until bar 16822 is positioned over the vertebral midline—as shown in FIGS. 14 and 15. A locking rod 173 is then placed into central bore of each tube 171 and is used to rigidly lock rod-receptacle member 164 relative to bone screw 166. In this way, platform 168 is rigidly affixed relative to the underlying vertebral bone. If desired, the platform 168 can be used to distract (or compress) the attached vertebral bones by actuating member 1682. Pawl 1684 retains the platform in the distracted position.

Instrument 130 is advanced through the skin and onto the lateral aspect of the anterior column of the target vertebral segment by the surgeon using free hand guidance and tactile feedback as previously described. Handle 132 of instrument 130 is removed by loosening threaded set screw 1322. The end of to member 1304 is then affixed to bar 16822 of platform 168—as shown in FIG. 18. A threaded set screw is placed through threaded bore 13044 of member 1304 and used to immobilize member 1304 relative to platform 168. (Note that the platform may be alternatively adapted to attach onto a bone screw assembly placed at each side vertebral midline. That is, at the target level of L3 and L4 vertebral bones, the platform may be adapted to attach onto a bone screw assembly placed within the right L3 pedicle, the left L3 pedicle, the right L4 pedicle and the left L4 pedicle.)

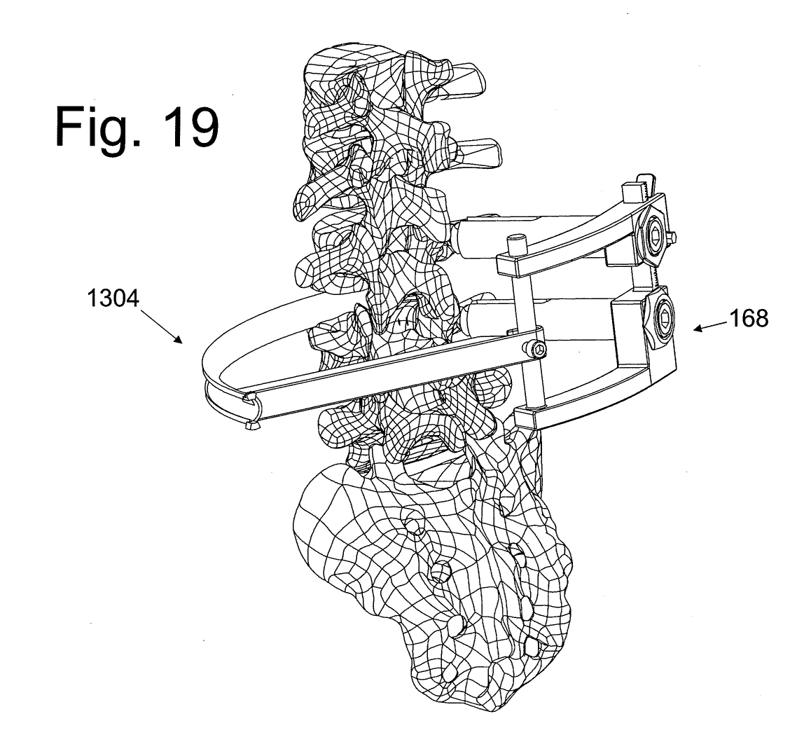

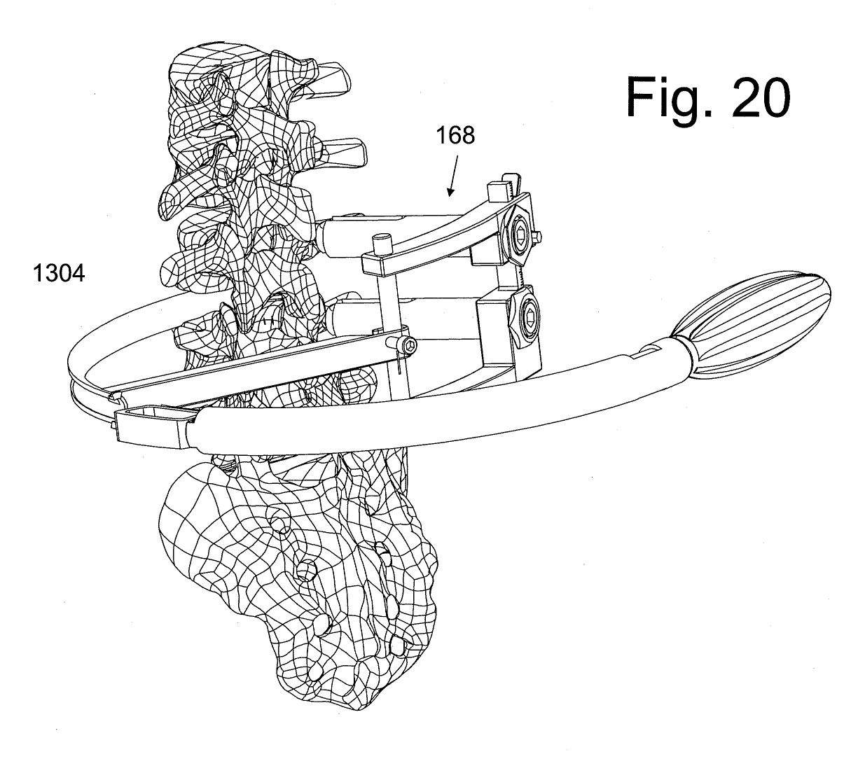

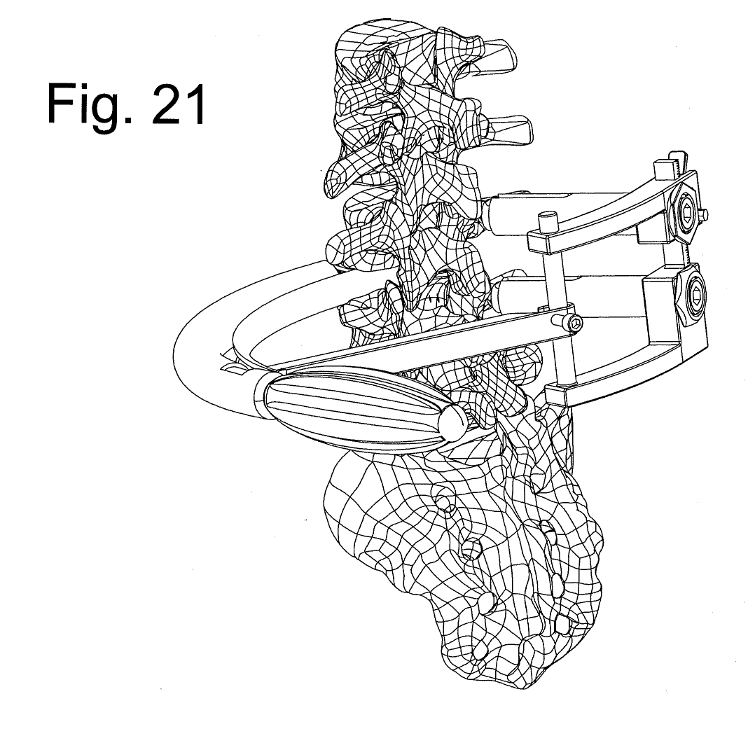

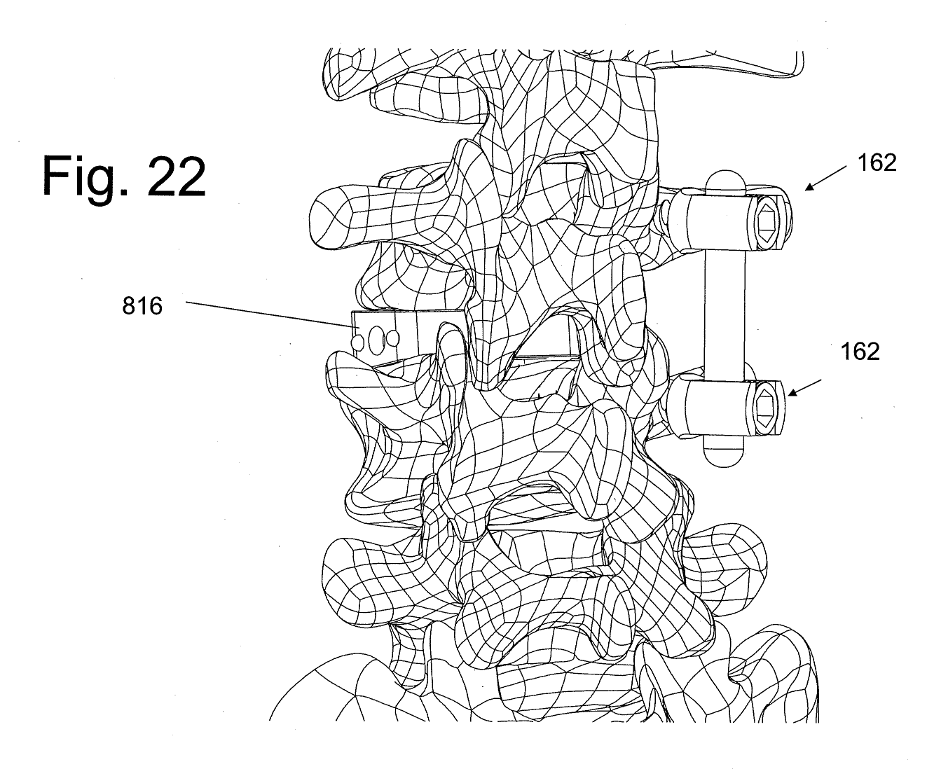

As shown in FIG. 19, device 505 is then removed and tract 13042 of member 1304 provides a tract along which work on the inter-vertebral disc space and/or lateral aspect of the vertebral bodies of the target spinal segment can be performed. An orthopedic implant can then be passed into (or onto) the spinal segment. An example of implantation of an orthopedic device is shown in FIGS. 20 and 21. After implant placement, the implant delivery instrument, member 1304 and platform 168 are removed. Locking rods 173 are removed and tubes 171 are used to deliver an interconnecting rod to the rod-receptacle members 164 of screw assemblies 162—as is well known in the art. Locking nuts are used to immobilize each of the L3 and L4 screw assemblies to the rod. The completed construct is shown in FIG. 22. (While not shown, a screw/rod assembly may be also placed within the contra-lateral pedicles of L3 and L4 so as to provide bilateral fixation of the L3 and L4 vertebral segments.)

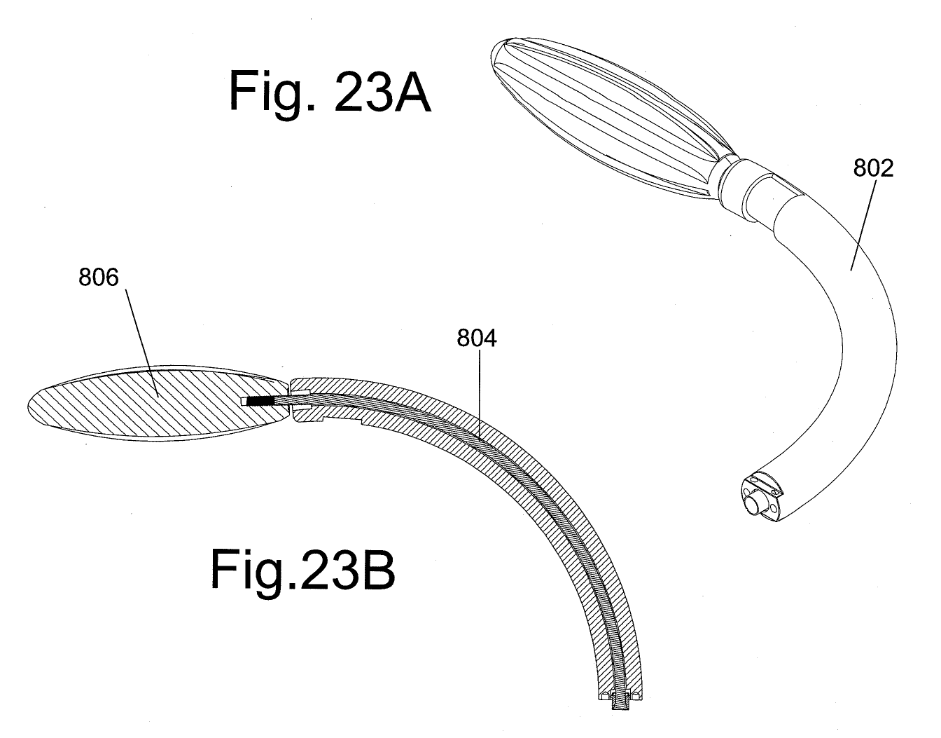

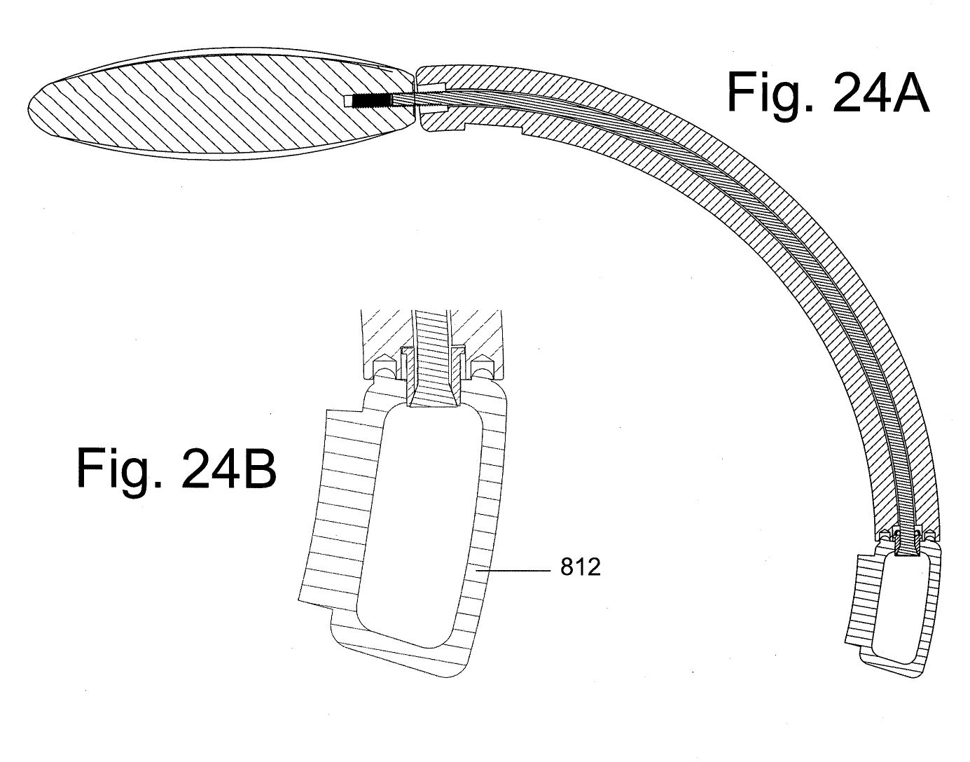

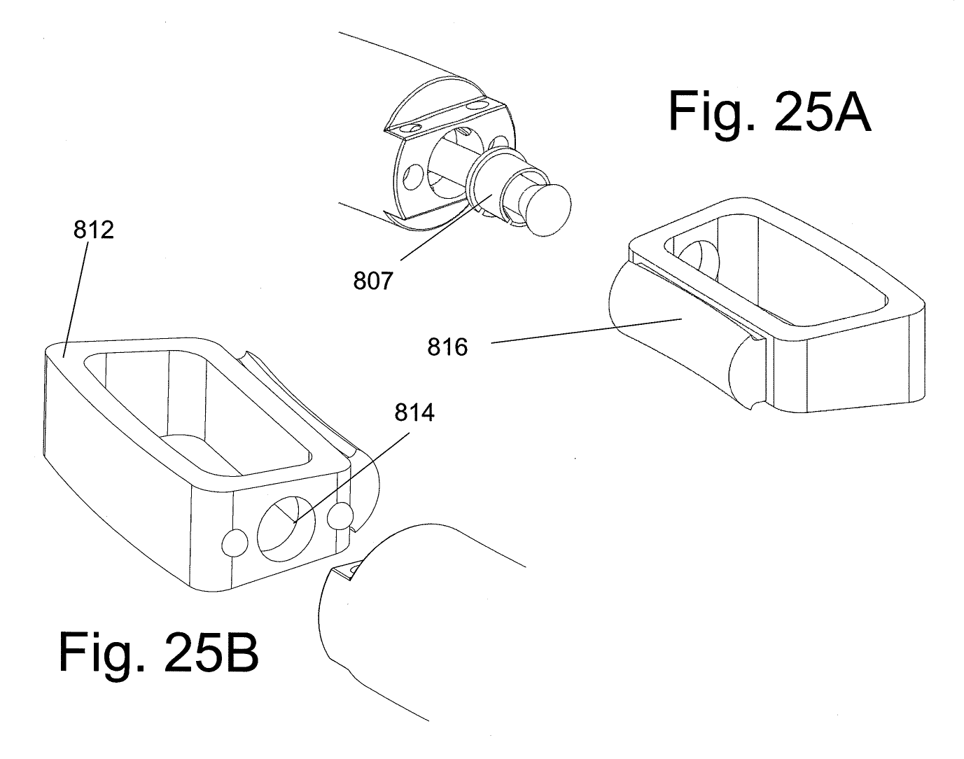

An implant placement instrument is illustrated in FIGS. 23A-23B and shown coupled to an implant in FIGS. 24A-24B. The attachment surface and mechanism of attachment are shown in FIGS. 25A-25B. The placement instrument has an outer curved cylindrical member 802 with inner member 804. The proximal end of member 804 is straight and threaded. Handle 806 contains a threaded bore that is adapted to interact with the threads of member 804. An expandable ring 807 (separate member) is located at the distal end of member 804 so that with rotation of handle 806, member 804 is moved towards handle 806 by the action of the interacting threads. The movement of member 804 produces forced expansion of the ring 807. Implant 812 has bore hole 814. When coupled to the placement instrument, the expansion of ring 807 produces a force onto the walls of bore 814 and immobilizes the implant relative to the placement instrument.

The implant contains side feature 816, wherein the feature interacts (and fits within the channel of) with the tract of the curvilinear guide member 1304 of instrument 130. While the implant is shown as bone graft containing implant adapted to promote bone fusion, it is understood that the implant is a generic illustration of any implant and/or substance that may be implanted into the inter-vertebral disc or bone (and/or onto the vertebral bone).

This method of use comprises a percutaneous and minimally invasive way to delivery an implant into or onto a desired segment of the spinal column. The target spinal segment is identified using radiographic imaging. The surgeon makes a skin incision that is lateral and posterior to the tip of the transverse process of the target spinal vertebrae, but not directly lateral to the target spinal segment. The body cavity is entered through the incision. A curved instrument with a curved side surface that is adapted to function as a guide rail for the advancement of other instruments is guided by the surgeon using tactile feel to the lateral aspect of the target spinal segment. (A finger may be used to palpate the internal aspect of the body cavity and guide the instrument to the target spinal segment.) A platform that had been previously positioned in a defined relationship to the target spinal segment is not used to guide the curved instrument to the target. The distal aspect of the curved port comes to rest anterior and medial to the tip of the transverse process and the location of the distal aspect is verified by x-ray visualization. The proximal aspect of the instrument is then attached to a platform with a defined spatial relationship to the target spinal segment, wherein the platform, when coupled to the instrument, limits the movement of the instrument relative to the targeted spinal segment. Surgical instruments are then advanced along the curved side surface of the instrument and onto the targeted spinal segment. The advanced surgical instruments are used to manipulate the target spinal segment and/or deliver implants or substances to the target segment.

FIG. 26 illustrates an additional method of implant placement. An instrument 610 with a convex lateral tract is positioned as shown in FIG. 26. (Note that instrument 130 has a convex tract.) A substantially straight tissue retractor is used to retract the tissues medial to instrument 610.

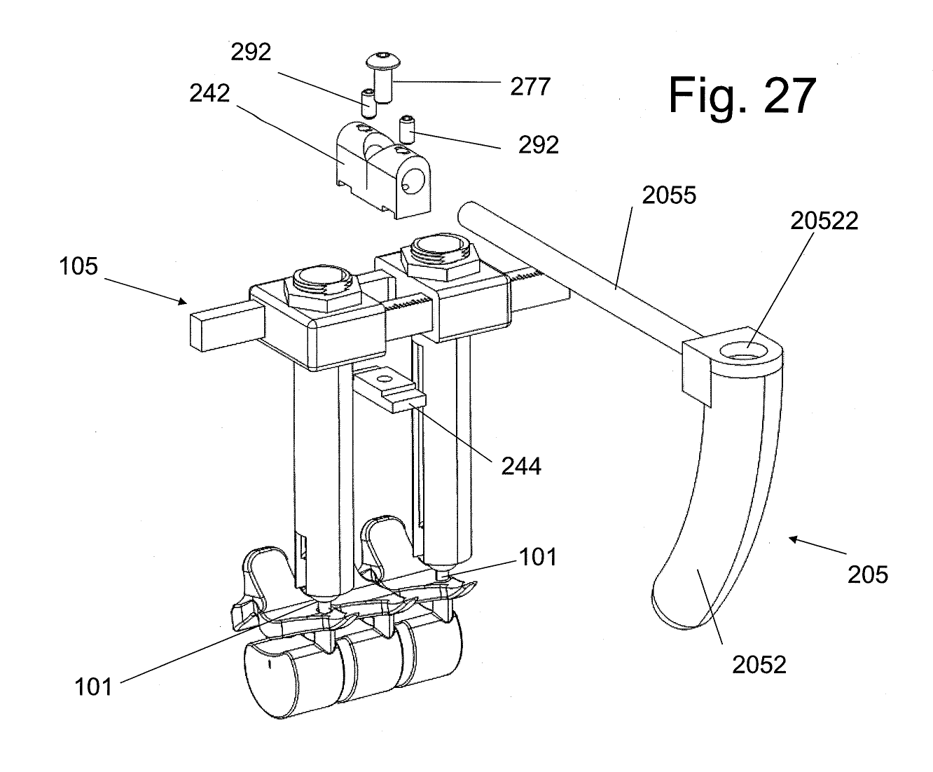

FIGS. 27 to 32 illustrate an additional embodiment. FIG. 27 shows a perspective view of a guide apparatus 205 which is affixed to the vertebral bones via bone screws 101. The vertebral bones of FIG. 27 are represented schematically and those skilled in the art will appreciate that actual vertebral bodies may include anatomical details that are not shown in FIG. 27. While the apparatus 205 is illustrated connecting to screws 101 via external cross-member 105, multiple other attachment embodiments may be alternatively employed by one of ordinary skill in the art. As shown, apparatus 205 contains a curvilinear tube member 2052 that has an internal aperture 20522 used to position an orthopedic implant onto the target spinal segment (such as the inter-vertebral disc and/or vertebral bodies). Preferably, curvilinear tube member 2052 has a radius R such that, when properly seated, the targeted spinal segment is included on the circumference of a circle passing through internal aperture 20522.

The device will be illustrated for use in targeting the inter-vertebral disc. Guide apparatus 205 is affixed to cross member 105 via upper seating member 242 and lower seating member 244. Threaded screw 277 (threads not shown) is used to affix the upper seating member onto the lower seating member and capture external cross-member 105 there between. Members 242 and 244 can travel relative to cross member 105 till screw 277 is locked. Before screw 277 lock, the location of the upper and lower members relative to the target spinal segment (i.e., disc space in this example) is ascertained by X-ray examination of the patient (preferably in at least two orthogonal planes). The upper and lower members are moved till they appropriately target the disc space. After screw 277 lock, members 242 and 244 form a rigid framework that is rigidly attached to member 105.

Rod member 2055 of apparatus 205 is inserted into aperture 2424 of the upper seating member 242. The rod can be adjustably rotated within aperture 2424 and can also translate in a medial/lateral. The guide apparatus 205 is positioned in an optimal position relative to the seating members so that an instrument placed through internal aperture 20522 of tube member 2052 is guided to the disc space. In an embodiment, the optimal position is selected based on X-ray examination during device placement and templates that are superimposed upon the x-ray image. The surgeon iteratively moves the guide apparatus and repeats the x-ray examination until the guide apparatus and the disc space align on relative to the templates. In another embodiment, a curved rod is manually advanced to the disc space by the surgeon in a manner similar to the insertion of instrument 130 described above. Preferably, the radius of the curved rod is slightly less that that of bore 20522. While maintaining the distal end of the curved rod at the disc space, the proximal is placed into bore 20522 while the apparatus 205 is in a non-rigid state. The trajectory of the curved rod is used to position and align the apparatus so that that an instrument placed through internal aperture 20522 of tube member 2052 is guided to the disc space. The device is then rigidly immobilized and the curved rod is removed, leaving an aligned apparatus 205 and an ready curvilinear tissue corridor (where the curved rod had been) that is adapted to accept interments adapted to manipulated the target disc pace

In order to lock apparatus 205, threaded set screws 292 (threads not shown) are advanced and rod member 2055 is rigidly immobilized relative to upper and lower seating members. In this way, the guide apparatus 205 is rigidly anchored to the underlying bones through the action of at least one affixed bone screw.

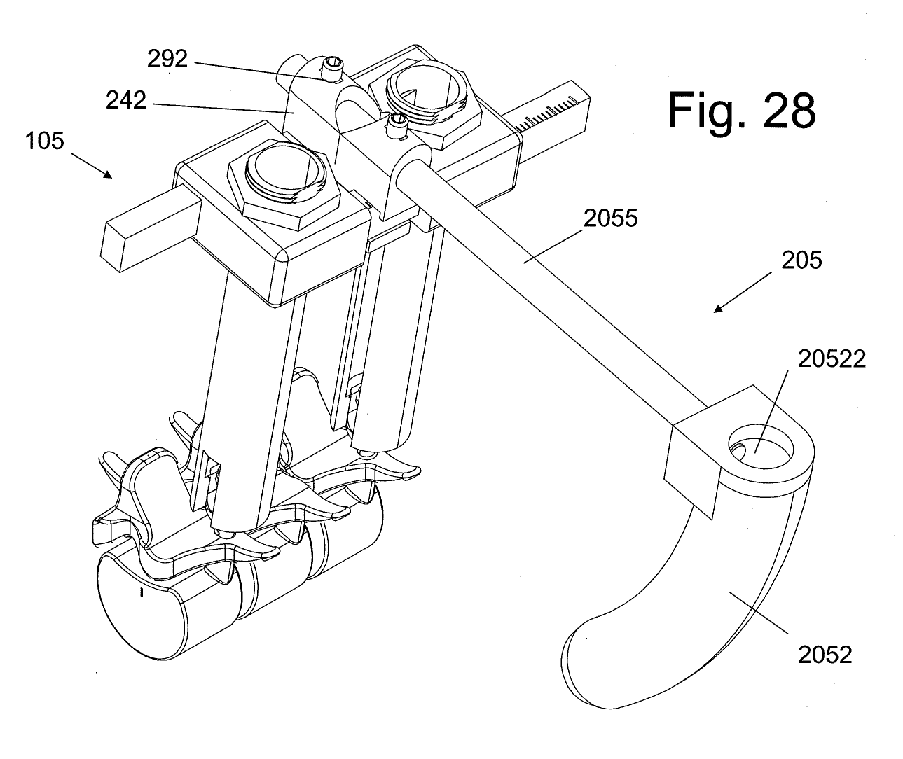

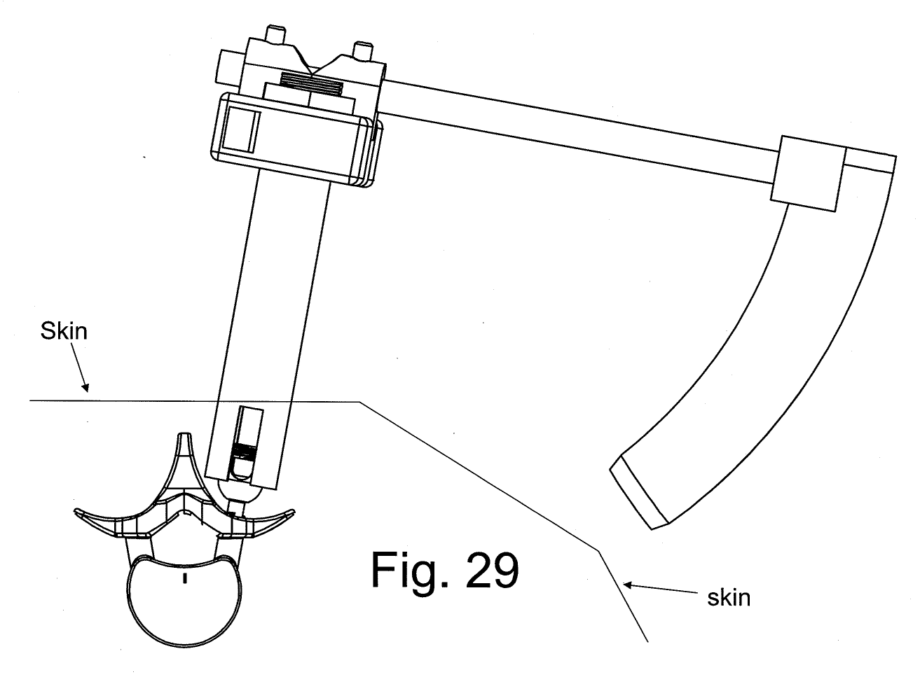

The assembled apparatus is shown in a perspective view in FIG. 28 and in a side view (i.e., in an axial plane relative to the patient) in FIG. 29. It is important to note that in the fully assembled state, guide apparatus 205 preferably remains outside of the skin. That is, in the preferred embodiment, guide apparatus 205 is not driven into the sub-cutaneous tissues of the patient but remains wholly outside of the patient's body. One of ordinary skill in the art will appreciate that skin and other anatomical features are not fully illustrated in the figures. Nevertheless, the illustrations intend to show that the mount assembly and guide rest outside of the subject body.

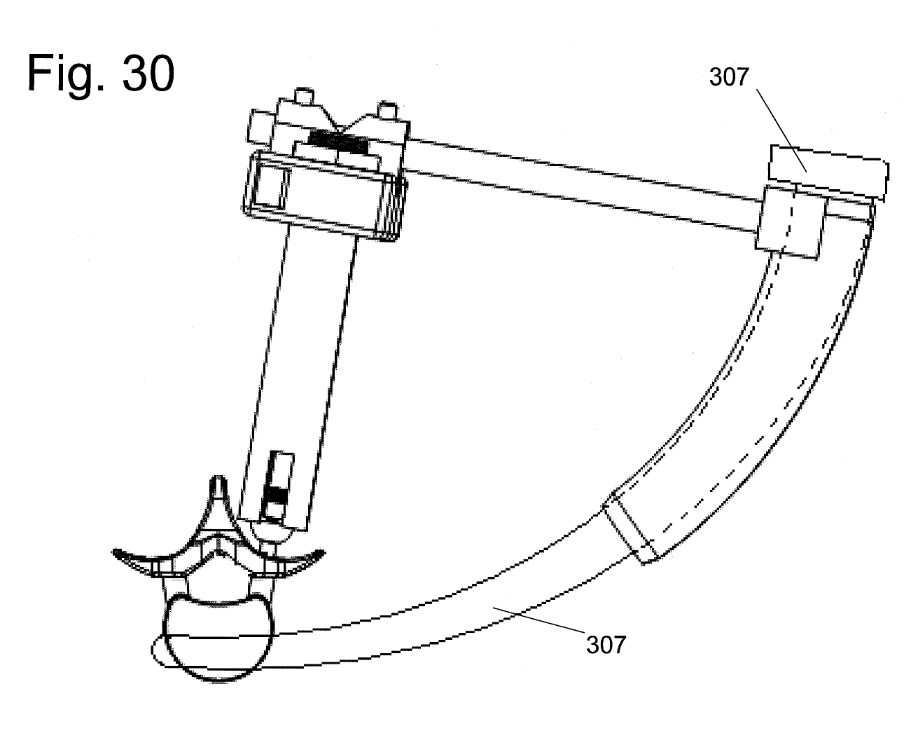

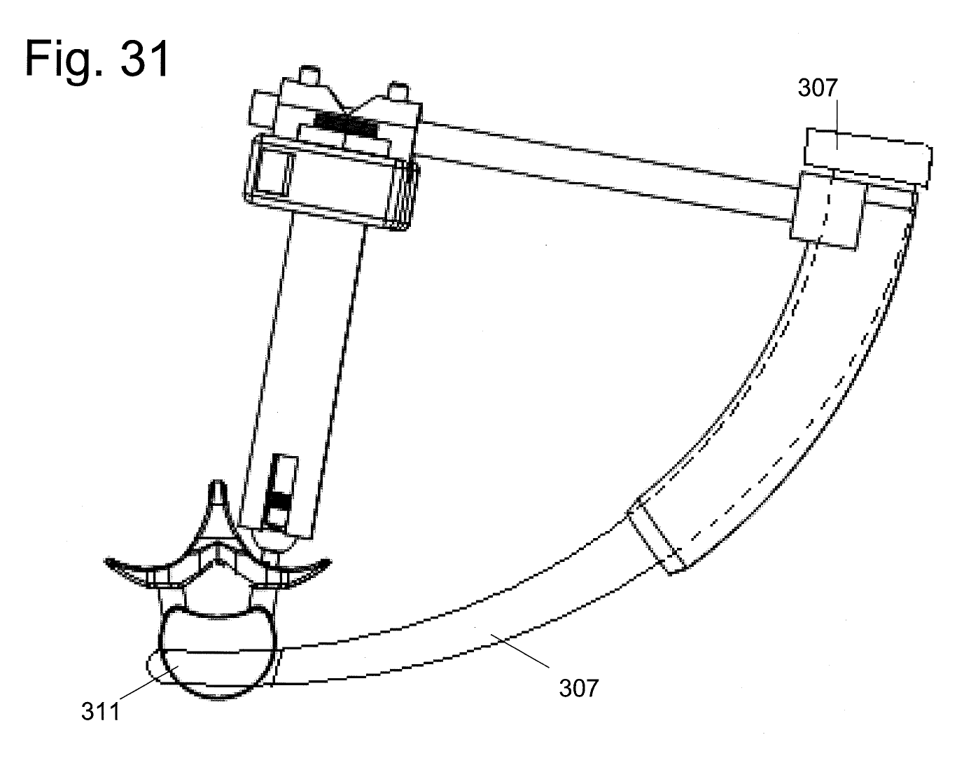

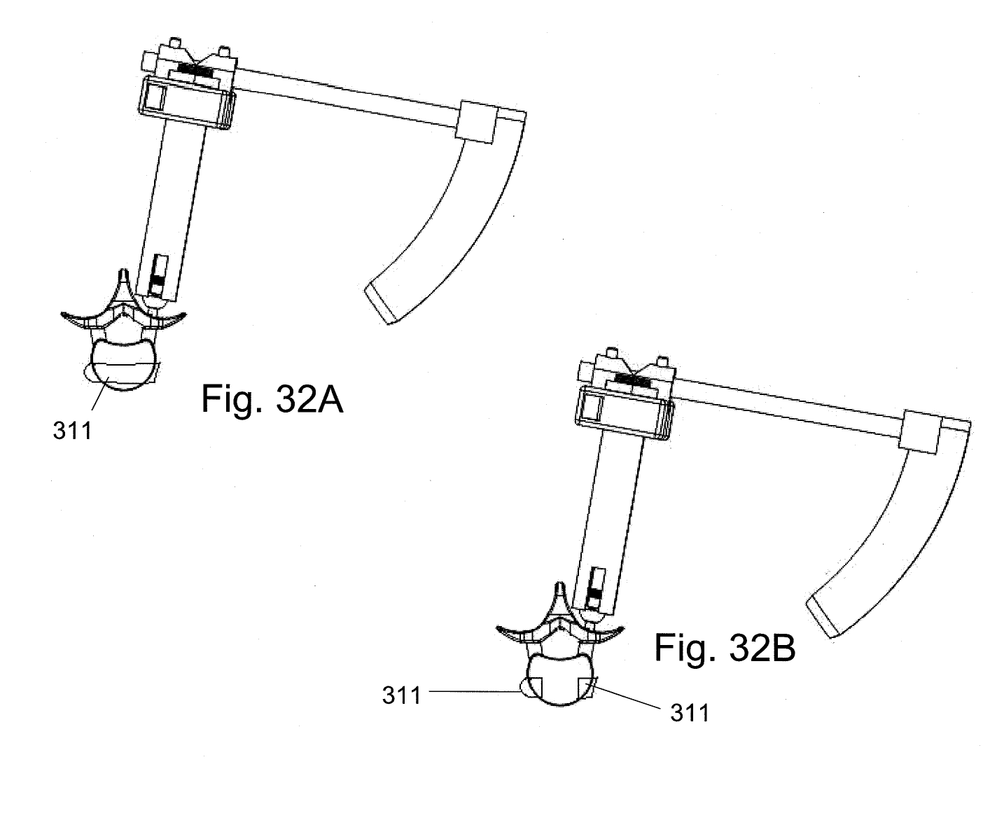

As shown in FIG. 30, a curvilinear instrument 307 is placed through internal aperture 20522 of tube member 2052 and advanced to the target spinal segment (such as, for example, the inter-vertebral disc space). Instrument 307 is used to percutaneously access the target. Once at the target, instrument 307 can also perform a variety of operations, including, but not limited to, perform a biopsy, remove disc and/or bony material, perform a discectomy, position an orthopedic implant into the disc space, and/or affix an orthopedic onto the vertebral bones that abut the targeted disc space. In an embodiment shown in FIG. 31, an instrument 307 is adapted to detachably couple to an orthopedic implant 311. For example, instrument 307 can be used to position implant 311 into the disc space. The instrument 307 is then detached form the implant and removed, leaving the implant within the disc space—as shown in FIG. 32A. By way of an additional example, FIG. 32B illustrates the impanation into the disc space of a more than one orthopedic implant 311. The multiple implants may be placed by repeated passes of instrument 307, wherein each pass places one implant, or by the attachment of multiple implants 311 onto instrument 307 and the positioning of multiple implants at a single pass of instrument 307.

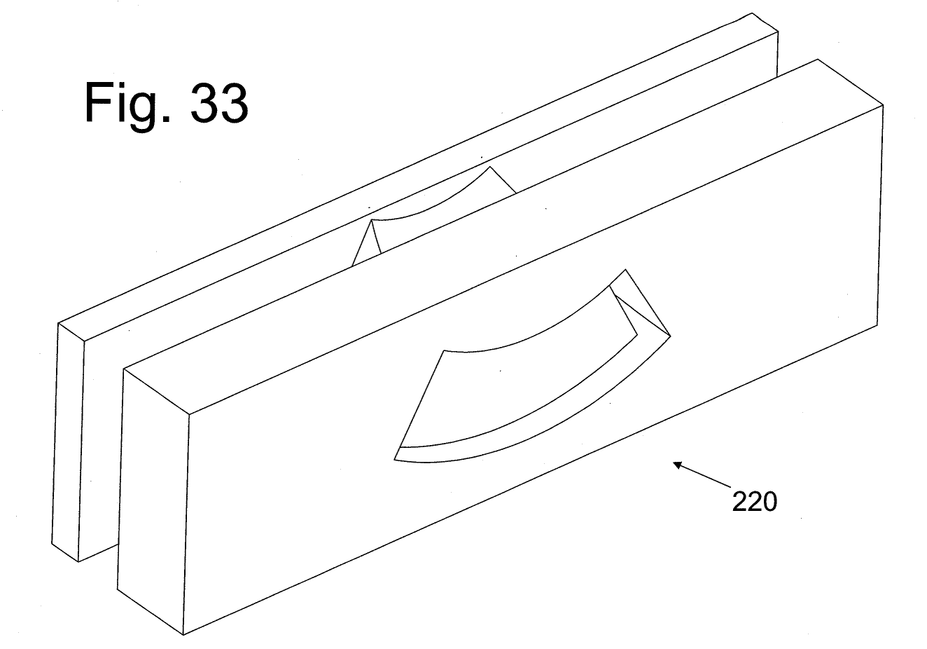

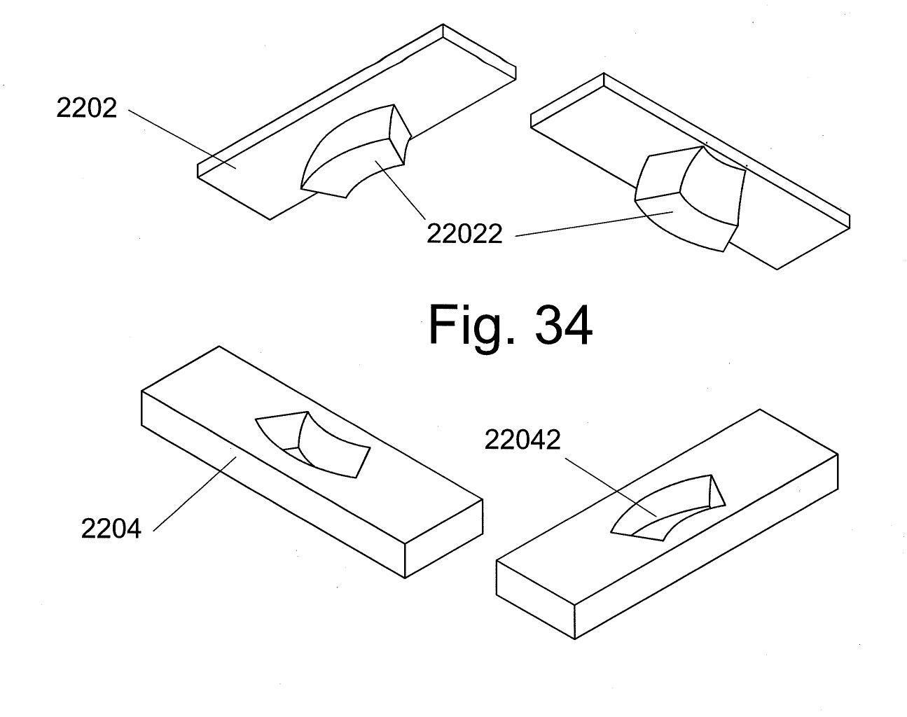

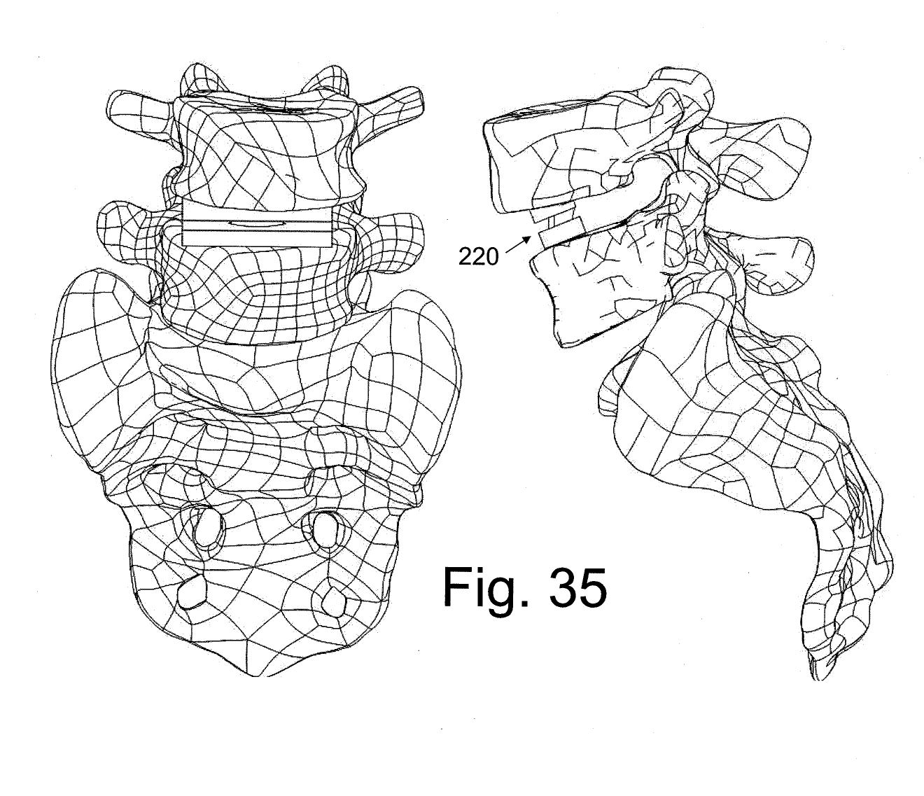

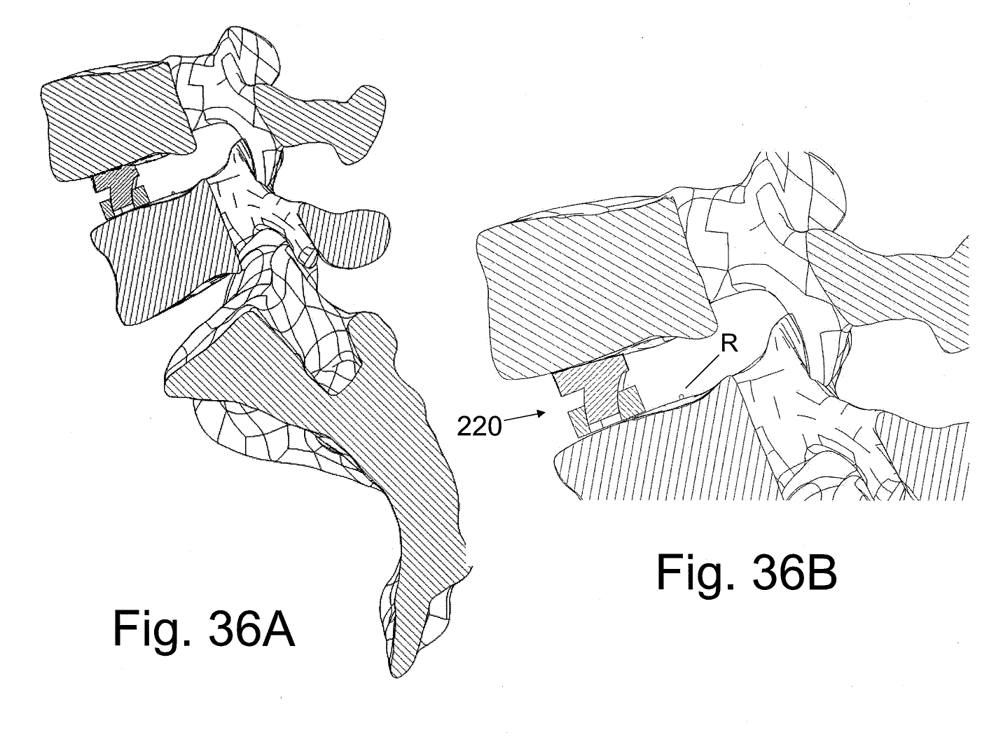

FIG. 33 illustrates an embodiment of a disc space prosthesis adapted to preserve motion between the two vertebras. Device 220 is an artificial disc implant capable of at least partially replicating the motion of the natural disc. FIG. 34 illustrates the upper member 2202 and lower member 2204 of device 220. The joint surface is formed by a spherical shell segment 22022 that protrudes from a surface of member 2202 and a complimentary cutout 22042 within member 2204. The implant permits movement about the center of the spherical joint surfaces wherein the center of rotation is preferably placed outside of the prosthesis. FIG. 35 shows the device in place. A mid sagittal cut of the spine with the implanted device is shown in FIG. 36A and a close-up view is shown in FIG. 36B. As shown, the center rotation R of the implant is posterior to the implanted device. While not shown, the prosthesis may have a keel member that protrudes form the bone-abutment surface of members 2202 and 2204 and is positioned within a corresponding cut placed into the adjacent bones. Prior to implantation of a keel-bearing implant, a bone cut is made in the vertebral surface on one or both sides of the disc space that is adapted to accommodate the keel.

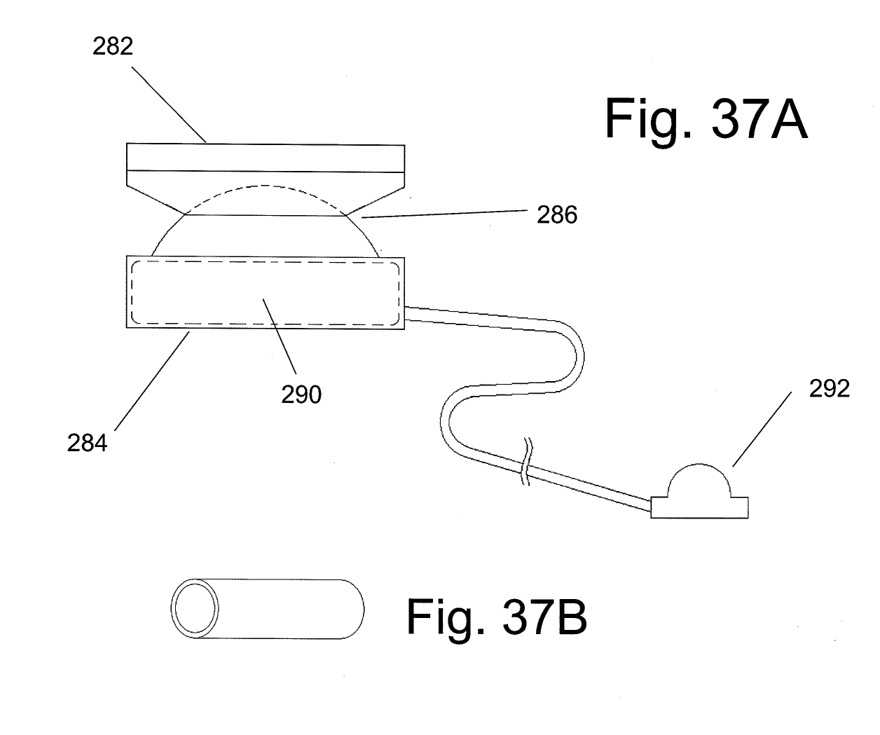

FIG. 37A illustrates a device adapted to provide an immediate replacement bearing surface within a damaged disc and also adapted to contain materials capable of regenerating, replacing and/or rejuvenating the disc over time. Surface 282 abuts the lower surface of the upper vertebral bone while surface 284 abuts the upper surface of the lower vertebra. A bearing surface 286 is located between surfaces 282 and 284. While depicted as a ball and socket articulation, any articulation surface configuration that is adapted to replace the function of the natural disc may be alternatively used. In an embodiment, the device contains a compartment 290 that is adapted to contain and house material(s) adapted to regenerate, replace and/or rejuvenate the function of the damaged disc. Such substances include, but are not limited to, one or more of the following: living or non-living biological cells, genetically engineered/altered genetic vectors (such as viruses and the like), extracts of biological tissues, natural or synthetic frameworks adapted to promote cell adhesion and/or growth, connective tissue matrices, nutrient-containing media, growth factors and the like. The compartment may be used to at least partially shield the contents from the loads present within the disc space. Alternatively, the compartment could be adapted to load the contents, as, for example, disclosed in US patent application 2006/0074488. The device may be made of any absorbable or non-absorbable material that is adapted for biological implantation. By way of example, use of an absorbable material would allow the device to serve a biological function at the time of implantation, such as disc replacement prosthesis, and then, at least partially reabsorb and permit the regenerated and/or replaced material generated by the substance(s) contained within compartment 290 to assume a biological function. Device re-absorption would also allow the regenerated and/or replaced disc to experience progressively more load as the prosthesis regresses over time and the regeneration/replacement product matures. The absorption rate may be linear or non-linear and may be specifically designed to affect the maturation of the regeneration/replacement product (for, example, by loading the product at a specified time(s) and/or by specified load amount(s)).

Further, compartment 290 may be accessed periodically in order to replenish and/or change the compartment contents. The compartment may be accessed directly or through a port 292 that is connected to it. Preferably, but not necessarily, the port is placed at a more accessible site (such as under the skin) away from the device. In another embodiment, the materials adapted to regenerate, replace and/or rejuvenate the disc may be coated directly onto the prosthesis and/or used as a constituent of the materials used to make the device. With time, at least a portion of the joint/bearing surface would be replaced with tissues (such as bone, cartilage, disc material, connective tissues and the like) that can function as a disc replacement and survive the loads present within the disc space. In these embodiments, a separate compartment 290 may not be needed. In another embodiment, the device may be coated with and/or made from osteo-inductive, osteo-conductive bio-active materials and/or bioactive factors that modulate tissue formation so as to promote tissue regeneration/formation and at least partially replace a portion of the device with bone and/or non-bony tissues.

In an embodiment, a tear of the disc annulus, whether natural or surgically created, may be repaired by delivering a device to either one or both sides of the defect, wherein the device is adapted to reinforce the tear and prevent disc fragments and/or other materials from traveling across it. Multiple devices for blockading an annular tear have been described in the art. In the current invention, a device adapted to reinforce the annulus is coated with, at least partially made from, and/or contains a compartment for the storage of a substance that is at least partially adapted to grow over time and regenerate, replace and/or rejuvenate the function of the disc annulus. Such substances include, but are not limited to, one or more of the following: living or non-living biological cells, genetically engineered/altered genetic vectors (such as viruses and the like), extracts of biological tissues, natural or synthetic frameworks adapted to promote cell adhesion and/or growth, connective tissue matrices, natural or synthetic frameworks adapted to assemble and/or after implantation, nutrient-containing media, growth factors and the like. In addition, the device may be made of an absorbable or non-absorbable material.

In another embodiment, an inflatable and/or expandable bag-like device is inserted into a disc space and filled with elastomeric substance. The surface of the device is made from an absorbable material and, with time, the surface dissipates to leave the elastomeric substance within the disc space. FIG. 37B illustrates a cylindrical implant with a central bore. The device is placed within a disc space and functions to shields the contents placed within the central bore from the loads that exist within the disc space. Soft tissue can be placed within the central bore and function to bring a vascularized tissue within the disc while simultaneously shielding that tissue from the intra-discal loads. Additional bores may be placed in the wall of cylindrical implant so that nutrients may transfuse from the vascular tissue within the central aspect of the device to the disc space tissues outside of the implant. In this way, the device functions to provide a vascular supply to the inner aspect of the disc space.

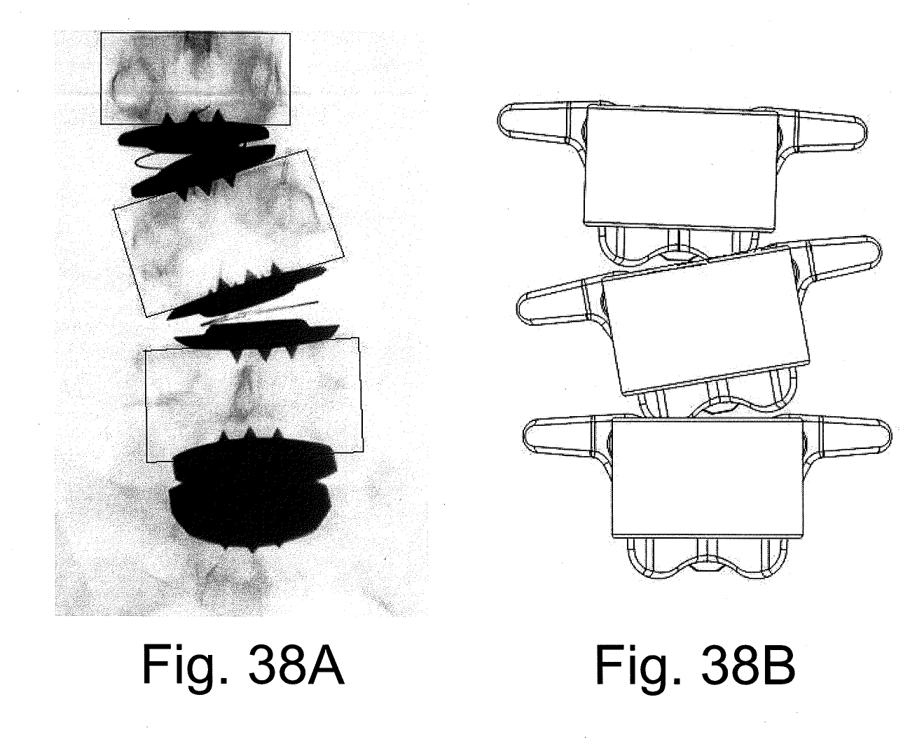

While the prior illustrations of an artificial disc prosthesis permit movement in all three plane, other embodiments can be created that permit movement in one or two planes alone. For example, the bearing surface of the implant could be shaped like a segment of a cylinder protrusion and a complimentary receptacle. In this embodiment, a snug-fitting receptacle surface would permit the prosthesis to flex and extend about the center of the cylinder but limit motion in other planes. Further, a less snug-fitting receptacle surface may permit movement in an additional plane but not movement in all three orthogonal planes. With placement of an artificial disc at multiple adjacent disc levels, the stacking of adjacent prosthesis's is likely to produce “snaking” and mal-alignment of the vertebral bones. The loss of spinal balance is particularly notable in the coronal plane, so that a scoliosis deformity may develop. FIG. 38A shows an X-ray from an actual patient, wherein the lumbar vertebrae have assumed an abnormal, scoliotic alignment as a direct result of the artificial disc implantation. FIG. 38B shows a schematic representation of the scoliotic deformity.

A method of multi-level artificial disc placement is disclosed. Deployment of the implantation method minimizes the likelihood of vertebral mal-alignment. Implant A is an artificial disc prosthesis permitting flexion/extension in the sagittal plane and limited or no lateral flexion in the coronal plane is employed. The prosthesis may or may not permit axial rotation. Implant B is an artificial disc prosthesis permitting lateral flexion in the coronal plane and limited or no flexion/extension in the sagittal plane. The prosthesis may or may not permit axial rotation. In use, no more than two adjacent disc spaces are implanted with the same implant type (i.e., either implant A or B). In an embodiment, for example, the implantation of three disc spaces would be performed by placing an implant A at the most inferior and most superior implanted space and an implant B in the middle implanted disc space. Preferably, the number of implants A used in the construct is equal to or greater than the number used of implants B.

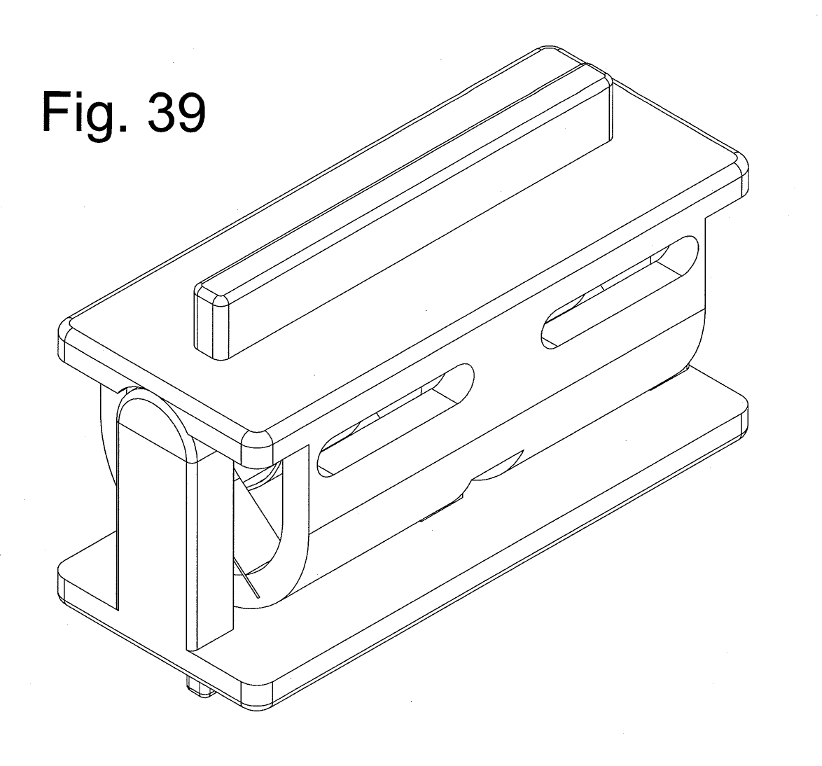

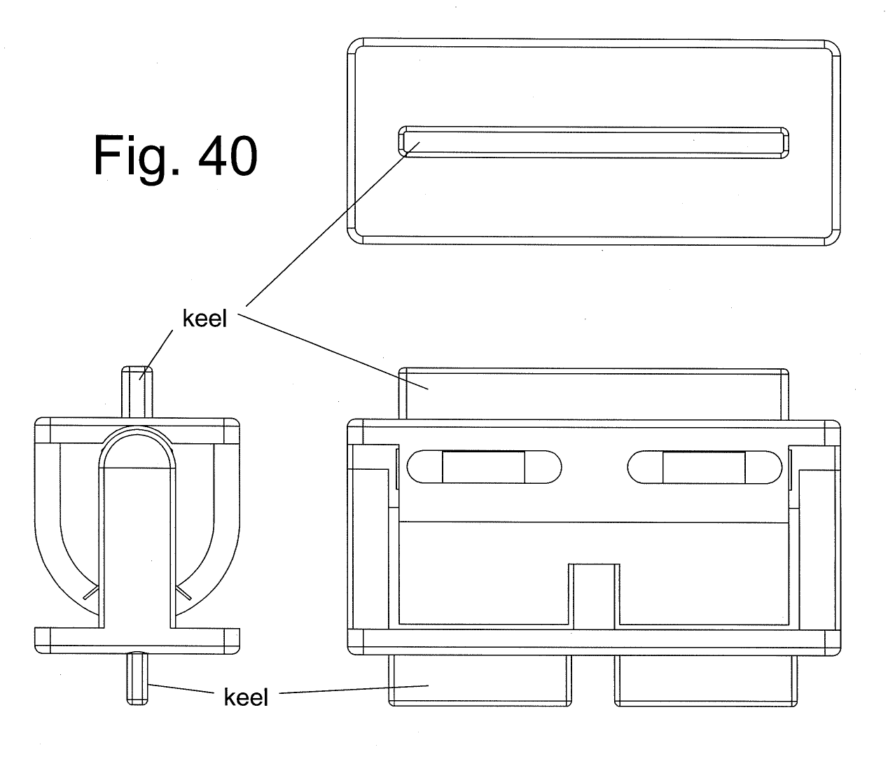

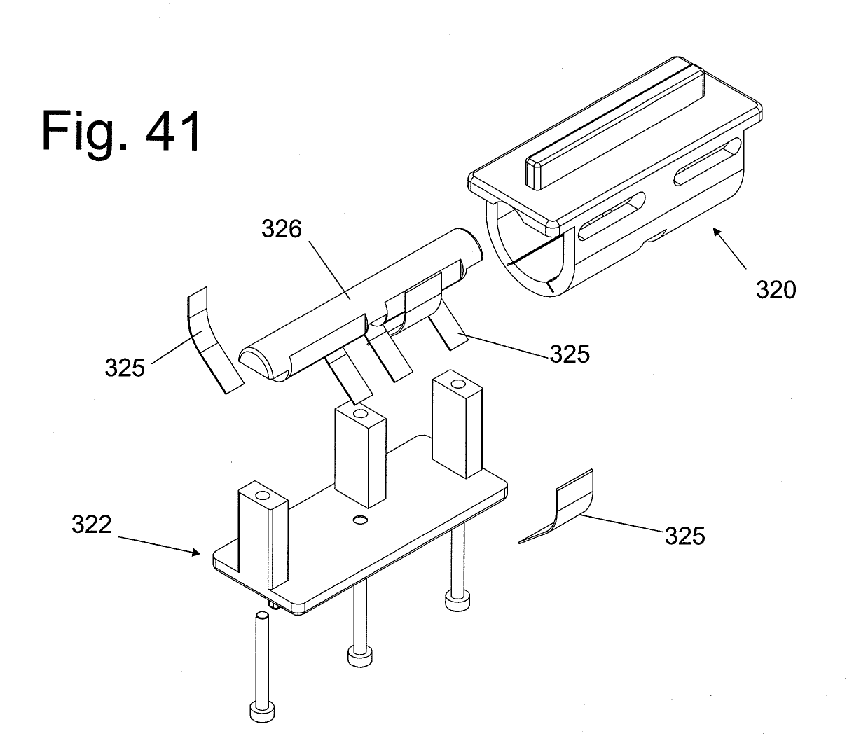

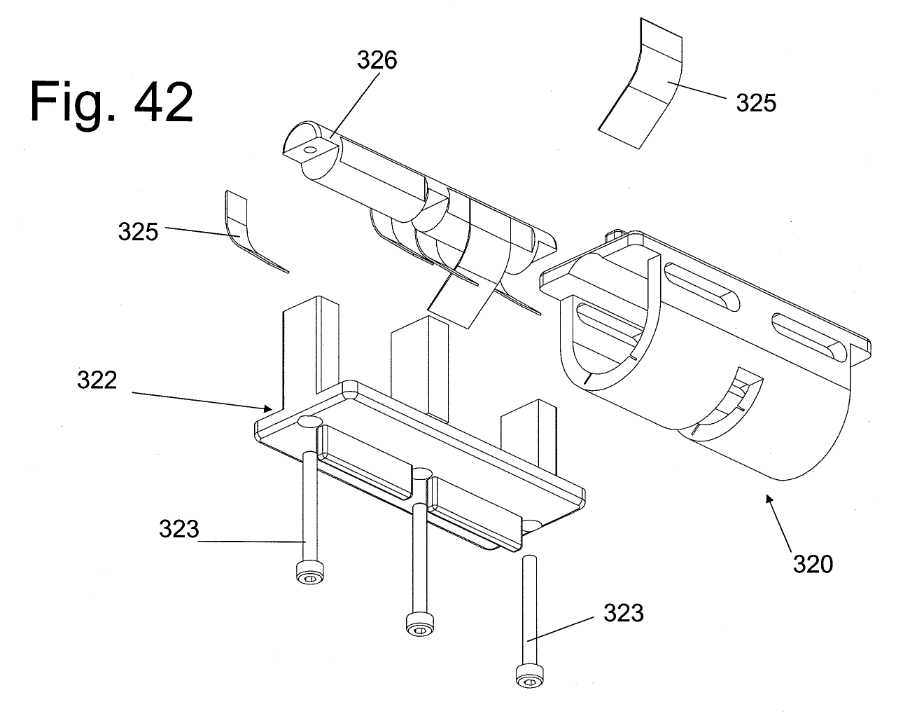

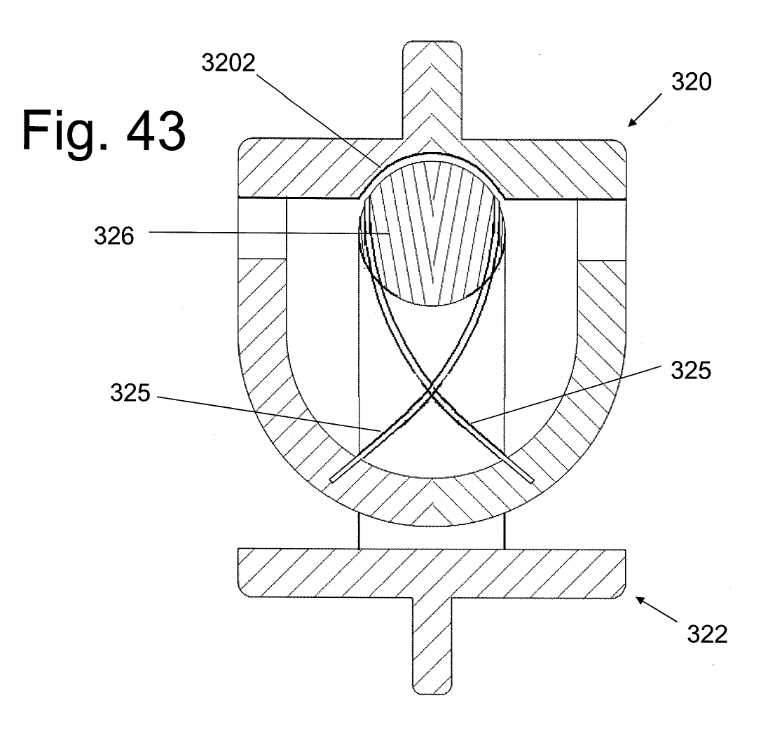

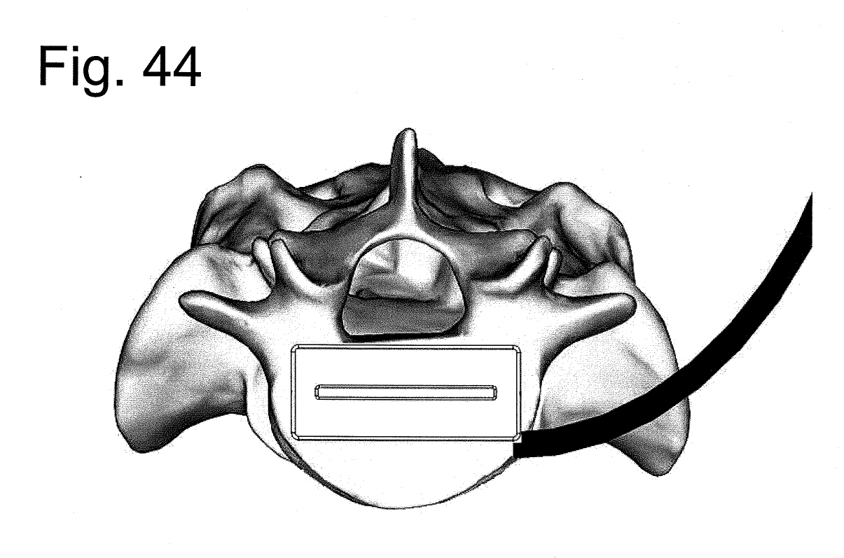

A device adapted to replace the function of an inter-vertebral disc is shown in FIG. 39. Multiple orthogonal views are shown in FIG. 40 while disassembled views are shown in FIGS. 41 and 42. A cross-sectional view is shown in FIG. 43. As shown, the device includes top segment 320 and bottom segment 322. Preferably, but not necessarily, a keel adapted to embed into and anchor onto a bone cut (placed by the surgeon within an abutting vertebral body) is located on at least one bone contact surface. In the assembled state, a substantially cylindrical member surface 326 is rigidly attached to bottom segment 322 (by members 323) and to malleable members 325 (via rigid or non-rigid attachment points). Each of members 325 is in turn attached to top segment 320. Member 326 is suspended within cut-out 3202 of top segment 320. The actions of malleable members 325 bias the devise towards a rest position (i.e., equilibrium configuration) and, with movement, members 325 will oppose motion away form equilibrium and act to restore the device to the rest position. FIG. 44 illustrates the device of FIG. 39 after lateral insertion into a disc space. The device of FIGS. 39-43 can be used to prevent the deformity illustrated on FIG. 38.

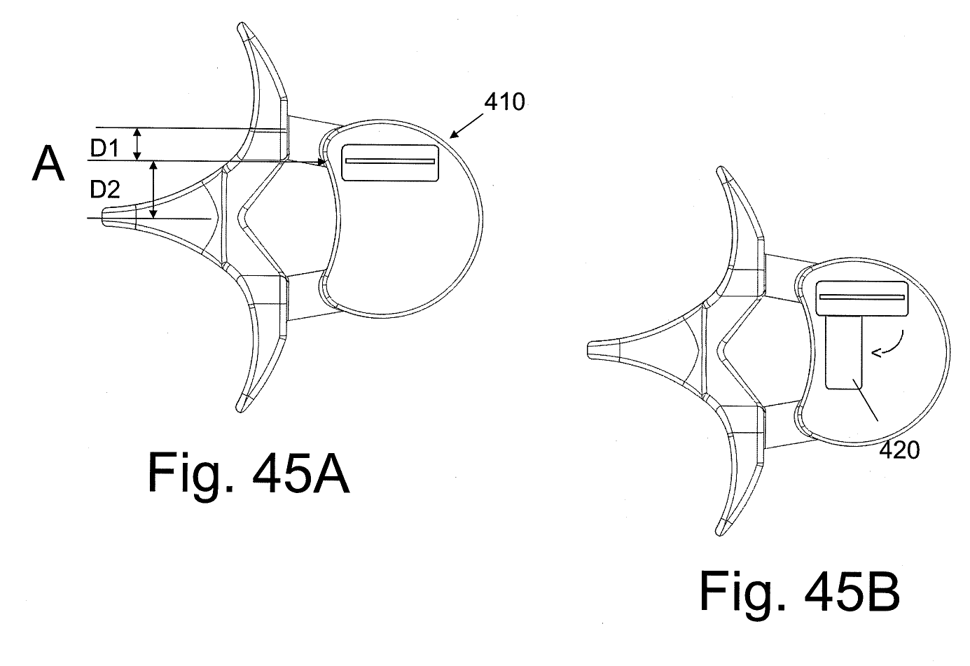

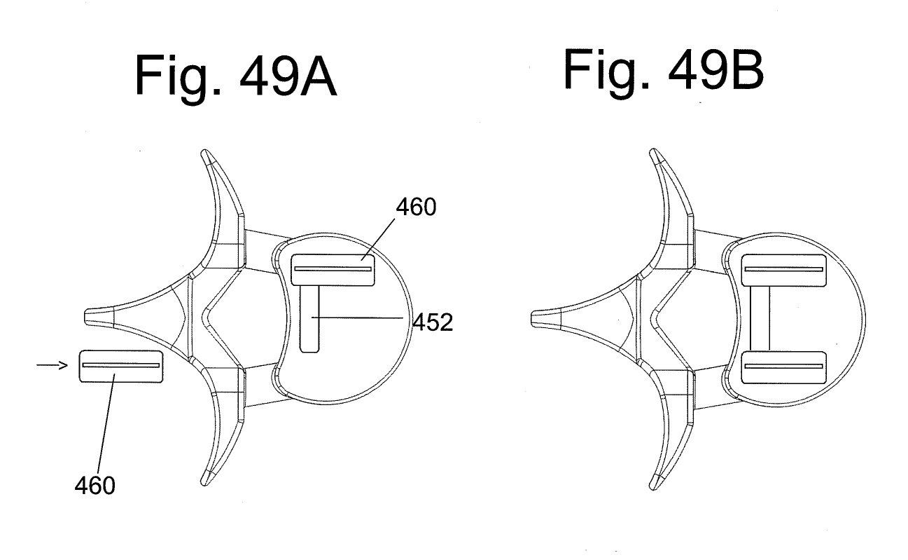

In an additional embodiment, the disc prosthesis may be repositioned within the disc space after insertion as shown in FIGS. 45A to 49B. While illustrated with insertion into the disc space through a posterior corridor, it is understood that this method of device reposition may be performed after use of other corridors (such as lateral or anterior) of initial device placement. A disc replacement prosthesis 410 is placed into the disc space form the direction illustrated by arrow “A” as shown in FIG. 45A. Preferably, the insertion site is selected based on the distance D1 from the adjacent pedicle (preferably of the lower vertebra), or the distance D2 from the spinous process of either vertebra (used as a marker of midline), or both. Basing the insertion site on distances D1 and/or D2 will insure proper orientation of the inserted implant relative to the disc space. In addition, these distances D1 and/or D2 may be used to insure symmetric implantation if an implant is placed on each side of the midline (as in, for example, FIG. 49).

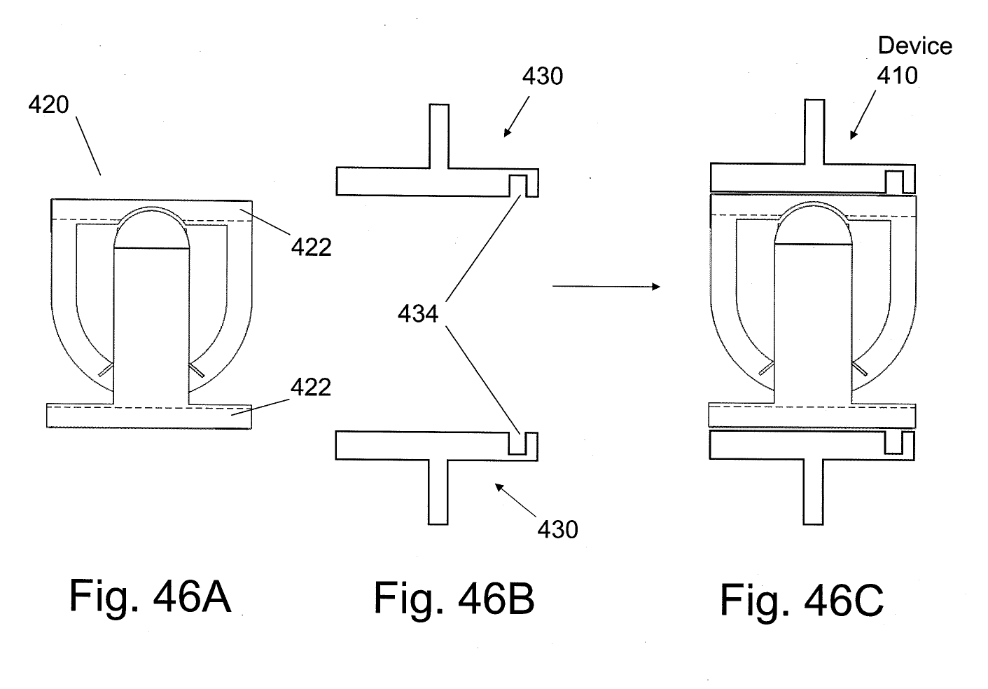

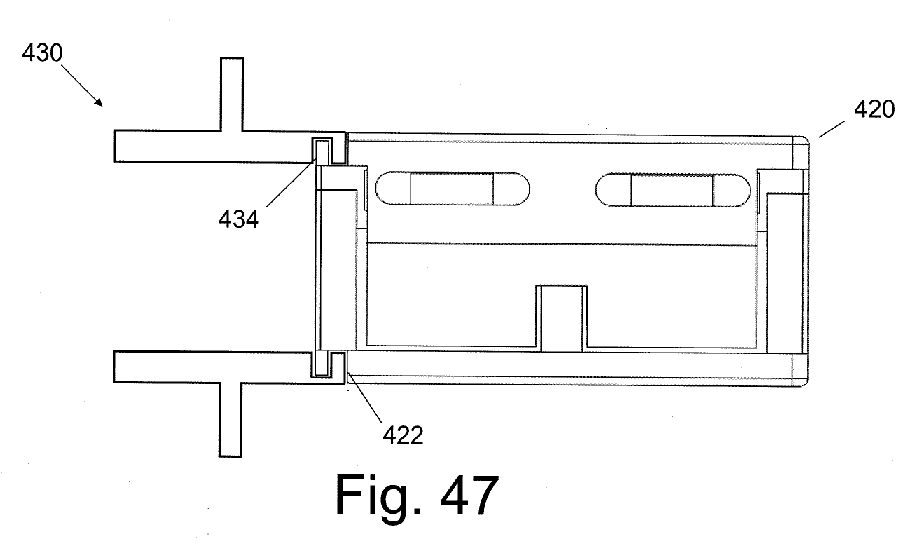

The segment 420 of the total device 410 that contains at least a portion of the mobile joint/bearing surface is then rotated, as shown in FIG. 45B. Preferably, after rotation, the segment 420 rests substantially within the posterior two thirds of the disc space. FIG. 46A shows a device embodiment wherein segment 420 is substantially similar to the embodiment of FIGS. 39-43. However, segment 420 does not contain keel member on the either bone abutment surface but does contain a channel 422 adapted to interact with segments 430 of total device 410. Segments 430 are shown in FIG. 46B and is each comprised of a bone abutment surface and a channel 434 adapted to interact with segment 420. Preferably, but not necessarily, a keel member protrudes from the bone abutment surface of segments 430 wherein each keel is adapted to fit within a cut bone channel in the vertebral surface that abuts member 430. The device is assembled before implantation and the assembled device is shown in FIG. 46C. After implantation, segment 420 is rotated relative to segment 420 and the device is re-configured to the device of FIG. 47. Note the interaction of channel 434 of segment 430 and channel 422 of segment 420. While the top and bottom bone abutment surfaces of segments 430 and 420 are shown as being co-planner and the same distance from the vertebral bone surface, they may alternatively rest at different distances from the bone. Finally, segment 420 maybe comprised of an upper and a lower bone abutment surfaces and a ball and socket joint between them (embodiment not shown).

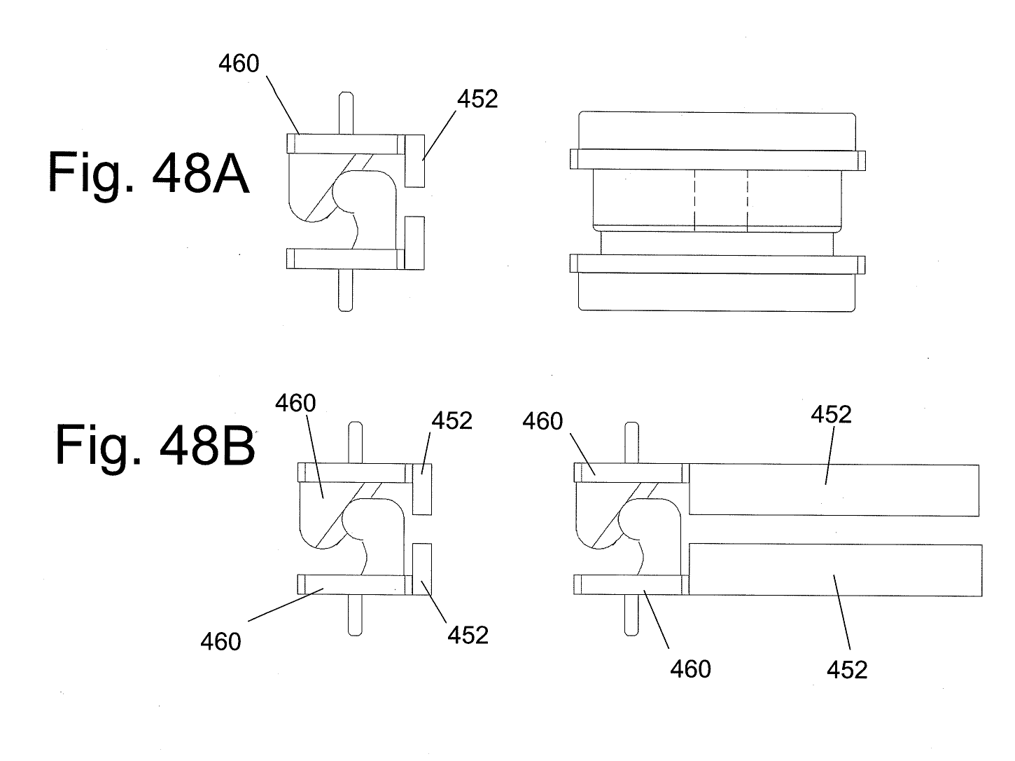

FIGS. 48A and 48B illustrate a device embodiment and method of placement wherein the rotated segment 452 does not contain a segment of the joint/articulation surface. The non-rotated segment 460 is comprised of at least a portion of the articulation surface, and, preferably, contains a keel protrusion on one or more of the bone abutment surfaces. Preferably, the implanted construct contains two segments 460 and one interconnecting segment 452 as shown in FIG. 49. An embodiment of segment 460 is shown in an end-on and side view in FIG. 48A. Note that segment 452 is attached to one side of segment 460. FIG. 48B shows an end-on view of member 452 and 460 with member 452 shown before rotation and after rotation.

While described as separate embodiments, the various mechanisms may be used in combinations to produce additional assemblies that have not been specifically described herein, but, nevertheless, would fall within the scope of this invention.

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable materials and the like. Any components may be also coated/made with nanotube materials to further impart unique mechanical or biological properties. In addition, any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, any surface may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. Lastly, the system or any of its components can also be entirely or partially made of a shape memory material or other deformable material.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A method for performing a procedure on a segment of a vertebral column of a subject, wherein the segments include at least two vertebral bones and an intervening disc space, comprising:
identifying a spinal disc space within the spinal segment using radiographic imaging, wherein the identified disc space is located between adjacent vertebrae and has an anterior aspect, a posterior aspect, a first side aspect and a second side aspect;
penetrating the skin of the subject at a position that is posterior to and lateral to the tip of the transverse process of the target spinal segment;
advancing an insertion member through the site of skin incision, wherein the insertion member comprises a curved elongate body that has a proximal end and a distal end, wherein the curved elongate body contains a curved outer tract that extends from a proximal end of the curved elongate body to a distal end of the curved elongate body;
forming an arcuate pathway from the skin incision site through tissues exterior to the vertebral column until the opening in the distal surface of the curved elongate body abuts the first side aspect of the target spinal segment;
coupling one end of a mount to the proximal end of the curved elongated body and another end of the mount to a surface with a defined spatial relationship to the identified disc space, wherein the coupled mount limits the movement of the insertion device relative to the disc space;
advancing an orthopedic device along the curved outer tract of the curved body member and onto the target spinal segment of the vertebral column.
